(19) United States
(12) Patent Application Publication
(10) Pub. No.: US 2018/0289637 A1
(43) Pub. Date: Oct. 11, 2018

(54) SUBSTITUTED CYCLOHEXANONES

(71) Applicant: CLEXIO BIOSCIENCES LTD., Jerusalem (IL)

(72) Inventors: Ralph Laufer, Tel Aviv (IL); Chengzhi Zhang, San Diego, CA (US); Yi Wang, Chester Springs, PA (US)

(21) Appl. No.: 16/004,679

(22) Filed: Jun. 11, 2018

Related U.S. Application Data
(63) Continuation-in-part of application No. PCT/US2017/026953, filed on Apr. 11, 2017.
(60) Provisional application No. 62/320,914, filed on Apr. 11, 2016.

(51) Int. Cl. A61K 31/135; A61K 9/00; A61P 25/24

(52) U.S. Cl. CPC A61K 31/135; A61P 25/24; A61K 9/0053

(57) ABSTRACT

The present disclosure provides compounds of Formula I and/or Formula II, or pharmaceutically acceptable salts thereof:

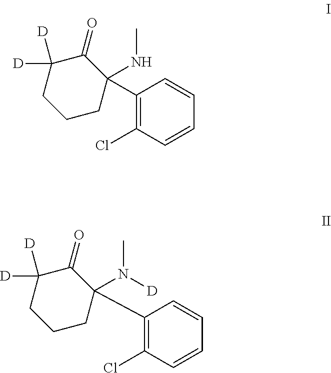

wherein D is deuterium and each deuterium has deuterium enrichment of no less than about 10%, compositions containing these compounds, and methods of using these compounds.

### CROSS REFERENCE TO RELATED APPLICATIONS

This application is a continuation-in-part of International Application No. PCT/US2017/026953, filed Apr. 11, 2017, which claims priority to U.S. Provisional Patent Application No. 62/320,914, filed Apr. 11, 2016, the entireties of which are incorporated herein by reference.

### FIELD

The present disclosure provides compounds of Formula I and/or II, or pharmaceutically acceptable salts thereof:

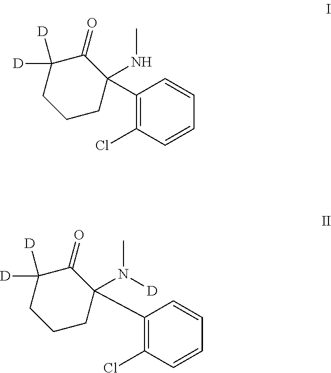

wherein D is deuterium; and each deuterium has deuterium enrichment of no less than about 10%, compositions containing these compounds, and methods of using these compounds.

### BACKGROUND

Ketamine is a racemic mixture of S-ketamine and R-ketamine. It is classed as a Schedule III Controlled Substance due to its potential for physical and psychological dependence, as well as its potential for abuse. At high doses, such as in Ketalor®, Ketaject®, and Ketalar®, it can be used as a general anesthetic. At subanesthetic doses (for example 0.2 mg/kg, 0.5 mg/kg), ketamine has been used experimentally, either intranasally or intravenously (IV), for the treatment of depression, specifically treatment resistant depression. However IV and intranasal ketamine, unlike current treatment options for depression, such as mono-amine oxidase inhibitors, tricyclic antidepressants, serotonin specific reuptake inhibitors, serotonin noradrenergic reuptake inhibitors, and noradrenaline reuptake inhibitors, produces a rapid antidepressant effect, acting within two hours and having an extended effect.

In addition to treatment for depression, human studies of low-dose ketamine for use in the treatment of Rett syndrome is being explored. Rett syndrome (RTT) is a rare genetic postnatal neurological disorder of the grey matter of the brain.

When administered orally, ketamine is subject to the first-pass liver metabolism via N-demethylation and conversion to the active metabolite N-desmethylketamine, usually referred to as norketamine. The elimination half-life of ketamine has been estimated at 2-3 hours, and 4 hours for norketamine. Due to extensive first pass metabolism which results in poor oral bioavailability, ketamine is typically administered parenterally or intranasally. Both of these routes of administration are inconvenient for a patient [Peltoniemi 2012, Basic & Clinical Pharmacology & Toxicology, 111, 325-332].

Oral administration of ketamine has been investigated to some extent (see Blonk, European Journal of Pain, 2010, 14, 466-472 and Fanta, European Journal of Clinical Pharmacology, 2015, 71, 441-447). Ketamine has been administered as an oral solution prepared from the commercially available injectable formulation (1 or 10% ketamine in water). Solid dose forms of ketamine have also been reported (Yanagihara, Biopharmaceutics & Drug Disposition, 2003, 24, 37-43) with pharmacokinetics in humans similar to the orally administered syrup formulation. Furthermore, oral and sublingual formulations of ketamine have been disclosed by Salama et al., WO 2014020155 and Chong 2009, Clinical Drug Investigation, 29(5), 317-324.

While ketamine is a racemic mixture of S-ketamine and R-ketamine, there is some controversy regarding the specific role of each enantiomer, as well as the mechanism of action. Ebert et al (1997) report that esketamine has a 5 times greater affinity for the NMDA receptor than (R)-ketamine, and (S)-norketamine has an 8 times higher affinity than (R)-norketamine in the inhibition of MK-801 binding. Oye et al (1992) report that esketamine was 4 times as potent as (R)-ketamine in reducing pain perception and in causing auditory and visual disturbances. Domino (2010) records that although esketamine appears more potent than (R)-ketamine, it also presents with greater undesirable psychotomimetic side effects. In contrast, Zhang et al (2014) and Yang et al (2015) have recorded that (R)-ketamine showed greater potency and longer-lasting antidepressant effects than esketamine in animal models of depression without psychotomimetic side effects and abuse liability. This has led some, such as Hashimoto (2016) to suggest that the anti-depressive effect of these molecules might not be due to NMDA receptor antagonism.

As such, there remains a need for more convenient, efficient, controllable, oral ketamine and ketamine-like products that mimic the results of ketamine IV for treatment of conditions such as pain, depression, traumatic brain injury, stroke, epilepsy, alcohol dependence, Rett or Alzheimer's disease.

### SUMMARY OF THE INVENTION

The present disclosure provides compounds of Formula I, or pharmaceutically acceptable salts thereof:

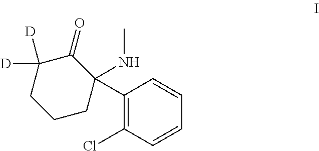

wherein D is deuterium and each deuterium has deuterium enrichment of no less than about 10%.

The present disclosure also provides compounds of formula II:

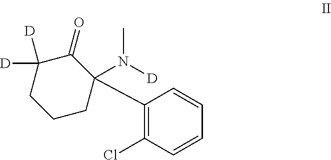

wherein, D is deuterium; and each deuterium has deuterium enrichment of no less than about 10%.

The present disclosure provides compositions comprising a compound of formula I and/or II or a pharmaceutically acceptable salt thereof. Further provided are pharmaceutical compositions comprising a compound of formula I and/or II or a pharmaceutically acceptable salt thereof, together with a pharmaceutically acceptable carrier.

The present disclosure provides methods for treating, preventing, or ameliorating one or more symptoms of disorders including, but not limited to alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder and the like using the compounds and compositions discussed herein.

Further provided is a compound of formula I and/or II or a pharmaceutically acceptable salt thereof, for use in treating a disorder. Further provided is a compound of formula I and/or II or a pharmaceutically acceptable salt thereof, for preparation of a medicament for treatment of a disorder. In a further embodiment of the compound or use, the disorder includes, but is not limited to a ketamine responsive disorder for example, alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, pain, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder and the like.

### BRIEF DESCRIPTION OF THE DRAWINGS

FIG. 1 shows the stability of the compound of Example 1 in phosphate-buffered saline, at the indicated solution pH and time points.

FIG. 2 shows the effect of D2, D6, and D8 esketamine compared to esketamine on Tail Suspension Test immobility in C57B1 mice treated i.p.

FIG. 3 shows the effect of D6, and D8 esketamine compared to esketamine in the Forced Swim Test in C57B1 mice treated i.p.

FIG. 4A shows mean immobility time during 5 mins (manual counting) in the Forced Swim Test in rats.

FIG. 4B shows mean immobility time during 5 mins (computerized counting) in the Forced Swim Test in rats.

FIG. 5 shows activity comparison of esketamine and D-2 esketamine in the Tail Suspension Test (6 minutes).

FIG. 6 shows mean immobility time in the Forced Swim Test in rats.

FIG. 7 shows the plasma concentration time profile for S-ketamine and a deuterated d2-S-ketamine compound of the disclosure after oral administration to rats at 60 mg/kg.

FIG. 8 shows the plasma concentration time profile for norketamine after oral administration of S-ketamine and a deuterated d2-S-ketamine compound to rats at 60 mg/kg.

FIG. 9 shows the plasma concentration time profile for 6-OH-norketamine after oral administration of S-ketamine and a deuterated d2-S-ketamine compound to rats at 60 mg/kg.

FIG. 10A depicts pharmacokinetics of esketamine dosed 15 mg/kg p.o. to rats.

FIG. 10B depicts pharmacokinetics of esketamine dosed 60 mg/kg p.o. to rats.

FIG. 11A depicts pharmacokinetics of D2-esketamine dosed 15 mg/kg p.o. to rats.

FIG. 11B depicts pharmacokinetics of D2-esketamine dosed 60 mg/kg p.o. to rats.

FIG. 12A depicts pharmacokinetics of D6-esketamine dosed 15 mg/kg p.o. to rats.

FIG. 12B depicts pharmacokinetics of D6-esketamine dosed 60 mg/kg p.o. to rats.

FIG. 13A depicts pharmacokinetics of D8-esketamine dosed 15 mg/kg p.o. to rats.

FIG. 13B depicts pharmacokinetics of D8-esketamine dosed 60 mg/kg p.o. to rats.

FIG. 14A depicts levels of 6-OH-norketamine (deuterated and non-deuterated) after administration of esketamine, D2-esketamine, D6-esketamine, and D8-esketamine dosed 15 mg/kg p.o. to rats.

FIG. 14B depicts levels of 6-OH-norketamine (deuterated and non-deuterated) after administration of esketamine, D2-esketamine, D6-esketamine, and D8-esketamine dosed 60 mg/kg p.o. to rats.

FIG. 15A depicts levels of dehydronorketamine (deuterated and non-deuterated) after administration of esketamine, D2-esketamine, D6-esketamine, and D8-esketamine dosed 15 mg/kg p.o. to rats.

FIG. 15B depicts levels of dehydronorketamine (deuterated and non-deuterated) after administration of esketamine, D2-esketamine, D6-esketamine, and D8-esketamine dosed 60 mg/kg p.o. to rats.

FIG. 16A depicts levels of esketamine (deuterated and non-deuterated) after administration of esketamine, D2-esketamine, D6-esketamine, and D8-esketamine dosed 15 mg/kg p.o. to rats.

FIG. 16B depicts levels of esketamine (deuterated and non-deuterated) after administration of esketamine, D2-esketamine, D6-esketamine, and D8-esketamine dosed 60 mg/kg p.o. to rats.

FIG. 17A depicts levels of norketamine (deuterated and non-deuterated) after administration of esketamine, D2-esketamine, D6-esketamine, and D8-esketamine dosed 15 mg/kg p.o. to rats.

FIG. 17B depicts levels of norketamine (deuterated and non-deuterated) after administration of esketamine, D2-esketamine, D6-esketamine, and D8-esketamine dosed 60 mg/kg p.o. to rats.

### DETAILED DESCRIPTION

To facilitate understanding of the disclosure set forth herein, a number of terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood in the art to which this disclosure belongs. In the event that there is a plurality of definitions for a term used herein, those in this section prevail unless stated otherwise.

Unless specifically defined otherwise, references to ketamine in this disclosure are to be understood to refer to racemic ketamine and/or its individual enantiomers, S-(esketamine) or R-ketamine.

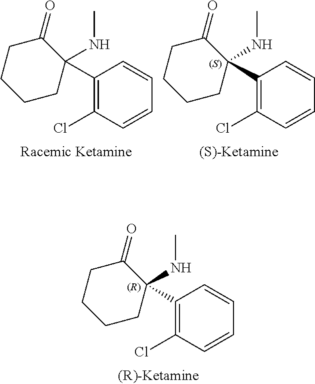

As used herein, “norketamine” or “N-desmethylketamine” are used interchangeably and have the below structure. Norketamine is an active metabolite of ketamine.

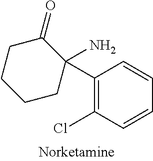 [SMILES: NC1(c2ccccc2Cl)CCCCC1=O]

“Hydroxynorketamine” discussed herein refers to 6-hydroxynorketamine, having the below structure, as well as its four stereoisomers. Hydroxynorketamine is a metabolite of ketamine.

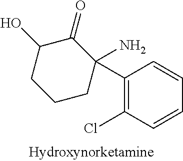 [SMILES: NC1(c2ccccc2Cl)CCCC(O)C1=O]

“Dehydronorketamine” discussed herein refers to the below structure, as well as its four stereoisomer. Dehydronorketamine is a metabolite of ketamine.

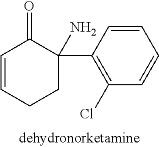 [SMILES: NC1(c2ccccc2Cl)CCC=CC1=O]

“D8-esketamine” refers to the below structure:

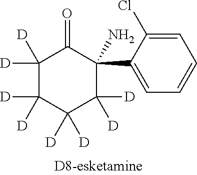 [SMILES: [2H]C1([2H])C(=O)[C@@](N)(c2ccccc2Cl)C([2H])([2H])C([2H])([2H])C1([2H])[2H]]

“D6-esketamine” refers to the below structure:

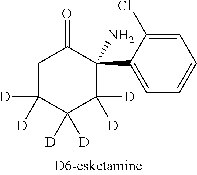 [SMILES: [2H]C1([2H])CC(=O)[C@@](N)(c2ccccc2Cl)C([2H])([2H])C1([2H])[2H]]

As used herein, the singular forms “a,” “an,” and “the” may refer to plural articles unless specifically stated otherwise.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, etc. The terms “subject” and “patient” are used interchangeably herein in reference, for example, to a mammalian subject, such as a human patient.

The terms “treat,” “treating,” and “treatment” are meant to include improving, preventing, alleviating or abrogating a disorder; or alleviating, preventing or abrogating one or more of the symptoms associated with the disorder; and/or preventing, alleviating or eradicating the cause(s) of the disorder itself, i.e., causing a clinical symptom to not significantly develop in a mammal that may be predisposed to the disease but does not yet experience or display symptoms of the disease. This may include improving the subject's ability to perform activities of daily living, perform domestic chores, manage finances, and/or perform an occupation or reduce the level of care needed by the subject. Treat, treating or treatment may include improvement of the symptom by at least 20%, 30%, 50%, 80%, 90%, or by 100%. Symptoms associated with a specific disorder depend on the specific disorder at hand. For example, in Rett syndrome, the symptom may be any one of more of the following: delay, partial or complete loss in acquiring mobility skills such as delayed or decreased motor coordination as in ability to sit, crawl, and/or walk; abnormal gait, ataxia, apraxia, muscle weakness, spasticity, rigidity; impaired gait initiation; abnormal muscle tone; hypotonia; peripheral vasomotor disturbance; scoliosis; delay, partial or complete loss in acquiring purposeful hand skills; abnormal hand movement such as wringing, squeezing, clapping, washing, tapping, rubbing, and/or repeatedly bringing hands to mouth; delay in acquiring communication skill such as a partial or complete loss of acquired communication skill such as eye contact, abnormal eye movement (staring, excessive blinking, crossed eyes, and/or closing one eye at a time); delay in acquiring language skill such as spoken language; breathing irregularity such as hyperventilation while may occur while awake as bruxism or while asleep as apnea. In one embodiment, the symptom is a breathing irregularity; increased irritability, decreased alertness, and/or decreased attention span; inappropriate laughing and/or screaming; seizure; cardiac abnormality such as bradycardia or tachycardia; decreased response to pain; growth retardation; microcephaly; impaired sleeping pattern; or hypotrophic cold blue feet.

“Treating” or “treatment” of a condition or disease includes: (1) preventing at least one symptom of the conditions, or (2) inhibiting the disease, i.e., arresting or reducing the development of the disease or its symptoms, or (3) relieving the disease, i.e., causing regression of the disease or its clinical symptoms. Treatment, prevention and ameliorating a condition, as used herein, can include, for example decreasing or eradicating a deleterious or harmful condition associated with Rett syndrome. Examples of such treatment include: decreasing breathing abnormalities, decreasing motor dysfunction, and improving respiratory and neurological function. The terms “prevent,” “preventing,” and “prevention” refer to a method of delaying or precluding the onset of a disorder; delaying or precluding its attendant symptoms; barring a subject from acquiring a disorder; and/or reducing a subject's risk of acquiring a disorder.

The term “therapeutically effective amount” refers to the amount of a compound that, when administered, is sufficient to prevent development of, alleviate to some extent or delay or prevent worsening of at least one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier” or “pharmaceutically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “deuterium enrichment” refers to the percentage of incorporation of deuterium at a given position in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods, such as mass spectrometry and nuclear magnetic resonance spectroscopy.

The term “is/are deuterium,” when used to describe a given position in a molecule or a drawing of a molecular structure, such as the symbol “D,” means that the specified position is deuterium or that the specified position is enriched with deuterium above the naturally occurring distribution of deuterium. In some embodiments, deuterium enrichment is no less than about 1%, in other embodiments, no less than about 5%, in further embodiments, no less than about 10%, in still other embodiments, no less than about 20%, in yet further embodiments, no less than about 50%, in other embodiments, no less than about 70%, in further embodiments, no less than about 80%, in yet other embodiments, no less than about 90%, or in still further embodiments, no less than about 98% of deuterium, at the specified position.

The term “non-isotopically enriched” refers to a molecule in which the percentages of the various isotopes are substantially the same as the naturally occurring percentages. For example, “non-isotopically enriched ketamine” refers to ketamine in which the percentages of the various isotopes, including deuterium, are substantially the same as the naturally occurring percentages.

The term “about” or “approximately” should be considered as disclosing the range defined by the absolute values of the two endpoints. The term “about” or “approximately” also means an acceptable error for a particular value, which depends in part on how the value is measured or determined. In certain embodiments, “about” can mean 1 or more standard deviations. For example, the expression “from about 2 to about 4” also discloses the range “from 2 to 4.” When used to modify a single number, the term “about” may refer to plus or minus 10% of the indicated number and includes the indicated number. For example, “about 10%” may indicate a range of 9% to 11%, and “about 1” means from 0.9-1.1.

The term “isomers” refers to different compounds that have the same molecular formula. The term “stereoisomers” refers to isomers that differ only in the way the atoms are arranged in space. The term “enantiomers” refers to stereoisomers that are non-superimposable mirror images of each other. A 1:1 mixture of a pair of enantiomers is a “racemic” mixture. The absolute stereochemistry is specified according to the Cahn-Ingold-Prelog R-S system.

The terms “active ingredient” and “active substance” refer to a compound, which is administered alone, or in combination with one or more pharmaceutically acceptable excipients and/or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “disorder” as used herein is intended to be generally synonymous, and is used interchangeably with, the terms “disease,” “syndrome,” and “condition” (as in medical condition), in that all reflect an abnormal condition of the body or one of its parts that impairs normal functioning and is typically manifested by distinguishing signs and symptoms.

The term “release controlling excipient” refers to an excipient having a primary function to modify the duration or place of release of the active substance from a dosage form as compared with a conventional immediate release dosage form.

The term “nonrelease controlling excipient” refers to an excipient having a primary function that does not include modifying the duration or place of release of the active substance from a dosage form as compared with a conventional immediate release dosage form.

The term “NMDA” refers to the N-methyl d-aspartate receptor. NMDA is a protein that facilitates the transport of ions, particularly calcium, sodium, and potassium, across certain cell membranes.

The term “AMPA” refers to the α-amino-3-hydroxy-5-methyl-4-isoxazolepropionic acid receptor. AMPA is a non-NMDA-type ionotropic transmembrane protein for glutamate that mediates fast synaptic transmission in the central nervous system.

The term “NMDA receptor-mediated disorder” refers to a disorder that is characterized by abnormal NMDA receptor (NMDAR) activity or normal NMDA receptor activity that, when that activity is modified, leads to the amelioration of other abnormal biological processes. An NMDA receptor-mediated disorder may be completely or partially mediated by the abnormal NMDA receptor. In particular, a NMDA receptor-mediated disorder is one in which modulation of the NMDA receptor activity results in some effect on the underlying disorder, e.g., an NMDA receptor modulator results in some improvement in at least some of the patients being treated.

The term “AMPA receptor-mediated disorder” refers to a disorder that is characterized by abnormal AMPA receptor (AMPAR) activity or normal AMPA receptor activity that, when that activity is modified, leads to the amelioration of other abnormal biological processes. An AMPA receptor-mediated disorder may be completely or partially mediated by the abnormal AMPA receptor. In particular, an AMPA receptor-mediated disorder is one in which modulation of the AMPA receptor activity results in some effect on the underlying disorder, e.g., an AMPA receptor modulator results in some improvement in at least some of the patients being treated.

The term “ketamine responsive disorder” refers to a disorder wherein the symptoms of the disorder can be alleviated by the administration of an effective amount of ketamine or wherein ketamine produces an effect on the subject.

The term “NMDA receptor modulator” or “modulation of NMDA receptors” refers to the ability of a compound disclosed herein to alter the function of an NMDA receptor. A modulator may activate the activity of an NMDA receptor, may activate or inhibit the activity of an NMDA receptor depending on the concentration of the compound exposed to the NMDA receptor, or may inhibit the activity of an NMDA receptor. Such activation or inhibition may be contingent on the occurrence of a specific event, such as activation of a signal transduction pathway, and/or may be manifest only in particular cell types. The term “NMDA receptor modulator” or “modulation of NMDA receptors” also refers to altering the function of an NMDA receptor by increasing or decreasing the probability that a complex forms between an NMDA receptor and a natural binding partner. A NDMA receptor modulator may increase the probability that such a complex forms between the NMDA receptor and the natural binding partner, may increase or decrease the probability that a complex forms between the NMDA receptor and the natural binding partner depending on the concentration of the compound exposed to the NMDA receptor, and or may decrease the probability that a complex forms between the NMDA receptor and the natural binding partner. In some embodiments, modulation of the NMDA receptor may be assessed using Receptor Selection and Amplification Technology (R-SAT) as described in U.S. Pat. No. 5,707,798, the disclosure of which is incorporated herein by reference in its entirety.

The term “AMPA receptor modulator” or “modulation of AMPA receptors” refers to the ability of a compound disclosed herein to alter the function of an AMPA receptor. A modulator may activate the activity of an AMPA receptor, may activate or inhibit the activity of an AMPA receptor depending on the concentration of the compound exposed to the AMPA receptor, or may inhibit the activity of an AMPA receptor. Such activation or inhibition may be contingent on the occurrence of a specific event, such as activation of a signal transduction pathway, and/or may be manifest only in particular cell types. The term “AMPA receptor modulator” or “modulation of AMPA receptors” also refers to altering the function of an NMDA receptor by increasing or decreasing the probability that a complex forms between an AMPA receptor and a natural binding partner. An AMPA receptor modulator may increase the probability that such a complex forms between the AMPA receptor and the natural binding partner, may increase or decrease the probability that a complex forms between the AMPA receptor and the natural binding partner depending on the concentration of the compound exposed to the AMPA receptor, and or may decrease the probability that a complex forms between the AMPA receptor and the natural binding partner. One skilled in the art would be able to utilize known assays to assess modulation of the AMPA receptor

The term “halide” or “halo” includes fluorine, chlorine, bromine, and iodine.

The term “alkyl” includes substituted, optionally substituted and unsubstituted C₁-C₁₀ straight chain saturated aliphatic hydrocarbon groups, substituted, optionally substituted and unsubstituted C₂-C₁₀ straight chain unsaturated aliphatic hydrocarbon groups, substituted, optionally substituted and unsubstituted C₂-C₁₀ branched saturated aliphatic hydrocarbon groups, substituted and unsubstituted C₂-C₁₀ branched unsaturated aliphatic hydrocarbon groups, substituted, optionally substituted and unsubstituted C₃-C₈ cyclic saturated aliphatic hydrocarbon groups, substituted, optionally substituted and unsubstituted C₅-C₈ cyclic unsaturated aliphatic hydrocarbon groups having the specified number of carbon atoms. For example, the definition of “alkyl” shall include but is not limited to: methyl (Me), trideuteromethyl (—CD₃), ethyl (Et), propyl (Pr), butyl (Bu), pentyl, hexyl, heptyl, octyl, nonyl, decyl, undecyl, ethenyl, propenyl, butenyl, pentyl, hexenyl, heptenyl, octenyl, nonenyl, decenyl, undecenyl, isopropyl (i-Pr), isobutyl (i-Bu), tert-butyl (t-Bu), sec-butyl (s-Bu), isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, adamantyl, norbornyl and the like.

The term “lower alkyl” means an alkyl having between 1 and 6 carbon atoms, i.e., C₁₋₆alkyl.

“Pharmaceutically acceptable salt” as used herein refers to a salt of a compound of the disclosure that is pharmaceutically acceptable and that possesses the desired pharmacological activity of the parent compound. Preferably, the salts are non-toxic may be inorganic or organic acid addition salts and base addition salts. In some embodiments, the salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentane propionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. In other embodiments, the salts are formed when an acidic proton is replaced by a metal ion, e.g., an alkali metal ion, an alkaline earth ion, or an aluminum ion; or coordinates with an organic base such as ethanolamine, diethanolamine, triethanolamine, N-methylglucamine and the like. In some embodiments, the salt contains one or more deuterium. In other embodiments, the salt is a DCl salt. In further embodiments, the salt is a HCl salt.

The term “prodrug” as used herein refers to a precursor of ketamine that, following administration to a subject, yields ketamine in vivo via a chemical or physiological process such as solvolysis or enzymatic cleavage, or under physiological conditions (e.g., a prodrug on being brought to physiological pH is converted to the compound of Formula I and/or II). Preferably, the prodrug is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985. In some embodiments, the prodrug is inter alia, an ester, glucuronide, or amino acid residue.

In some embodiments, the present disclosure provides compounds of Formula I, or pharmaceutically acceptable salts thereof:

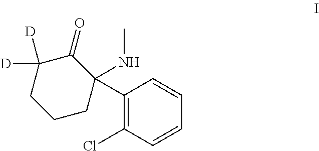

wherein, D is deuterium and each deuterium has deuterium enrichment of no less than about 10%.

In certain embodiments, the compound is d2-R-ketamine, or a pharmaceutically acceptable salt thereof:

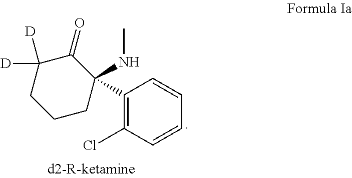 [SMILES: [2H]C1([2H])CCC[C@@](NC)(c2ccccc2Cl)C1=O]

In other embodiments, the compound is d2-S-ketamine, or a pharmaceutically acceptable salt thereof:

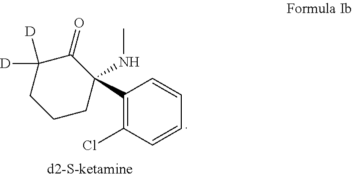 [SMILES: [2H]C1([2H])CCC[C@](NC)(c2ccccc2Cl)C1=O]

In further embodiments, the compound is a mixture of d2-R-ketamine:

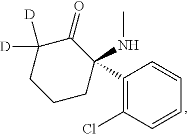 [SMILES: [2H]C1([2H])CCC[C@@](NC)(c2ccccc2Cl)C1=O]

or a pharmaceutically acceptable salt thereof; and d2-S-ketamine:

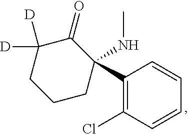 [SMILES: [2H]C1([2H])CCC[C@](NC)(c2ccccc2Cl)C1=O]

or a pharmaceutically acceptable salt thereof.

In yet further embodiments, the present disclosure provides compounds of formula II:

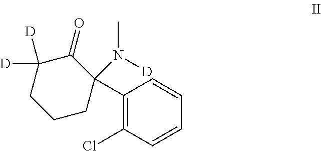

wherein, D is deuterium; and each deuterium has deuterium enrichment of no less than about 10%.

In certain embodiments, the compound is d3-R-ketamine, or a pharmaceutically acceptable salt thereof:

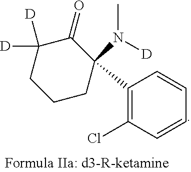

In other embodiments, the compound is d3-S-ketamine, or a pharmaceutically acceptable salt thereof:

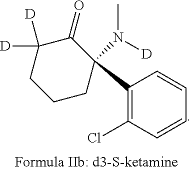

In further embodiments, the compound is a mixture of d3-R-ketamine:

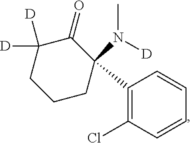

or a pharmaceutically acceptable salt thereof; and d3-S-ketamine:

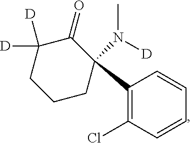

or a pharmaceutically acceptable salt thereof.

In still other embodiments, the compound is a mixture of d2-R-ketamine or a pharmaceutically acceptable salt thereof and d3-R-ketamine or a pharmaceutically acceptable salt thereof.

In yet further embodiments, the compound is a mixture of d2-S-ketamine or a pharmaceutically acceptable salt thereof and d3-S-ketamine or a pharmaceutically acceptable salt thereof.

In other embodiments, the compound is a mixture of d2-S-ketamine or a pharmaceutically acceptable salt thereof and d3-R-ketamine or a pharmaceutically acceptable salt thereof.

In further embodiments, the compound is a mixture of d2-R-ketamine or a pharmaceutically acceptable salt thereof and d3-S-ketamine or a pharmaceutically acceptable salt thereof.

In yet other embodiments, the compound is a mixture of d2-S-ketamine or a pharmaceutically acceptable salt thereof, d2-R-ketamine or a pharmaceutically acceptable salt thereof, and d3-S-ketamine or a pharmaceutically acceptable salt thereof.

In still further embodiments, the compound is a mixture of d2-S-ketamine or a pharmaceutically acceptable salt thereof, d2-R-ketamine or a pharmaceutically acceptable salt thereof, and d3-R-ketamine or a pharmaceutically acceptable salt thereof.

In other embodiments, the compound is a mixture of d3-R-ketamine or a pharmaceutically acceptable salt thereof, d3-S-ketamine or a pharmaceutically acceptable salt thereof, and d2-S-ketamine or a pharmaceutically acceptable salt thereof.

In further embodiments, the compound is a mixture of d3-R-ketamine or a pharmaceutically acceptable salt thereof, d3-S-ketamine or a pharmaceutically acceptable salt thereof, and d2-R-ketamine or a pharmaceutically acceptable salt thereof

In yet other embodiments, the compound is a mixture of d2-R-ketamine or a pharmaceutically acceptable salt thereof, d2-S-ketamine or a pharmaceutically acceptable salt thereof, d3-R-ketamine or a pharmaceutically acceptable salt thereof, and d3-S-ketamine or a pharmaceutically acceptable salt thereof.

In further embodiments, each compound of Formula I, Ia, Ib, II, IIa, or IIb is a free base.

In other embodiments, each compound of Formula I, Ia, Ib, II, IIa, or IIb is a pharmaceutically acceptable salt. In some preferred embodiments, the compound is an HCl salt of Formula I, Ia, Ib, II, IIa, or IIb. In other preferred embodiments, the compound is a DCl salt of Formula I, Ia, Ib, II, IIa, or IIb.

In preferred embodiments, the compound of Formula Ia or Ib is a hydrogen chloride salt of d2-R-ketamine, d2-S-ketamine, or mixtures thereof:

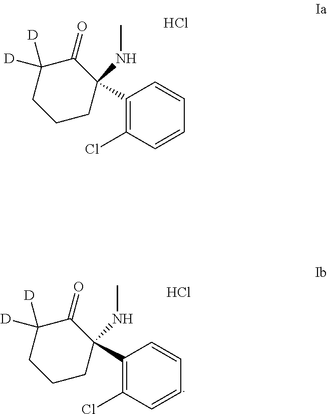 [SMILES: Cl ; [2H]C1([2H])CCC[C@@](NC)(c2ccccc2Cl)C1=O ; [2H]C1([2H])CCC[C@](NC)(c2ccccc2Cl)C1=O ; Cl]

In other preferred embodiments, the compound of Formula IIa or IIb is a hydrogen chloride salt of d3-R-ketamine, d3-S-ketamine or mixtures thereof:

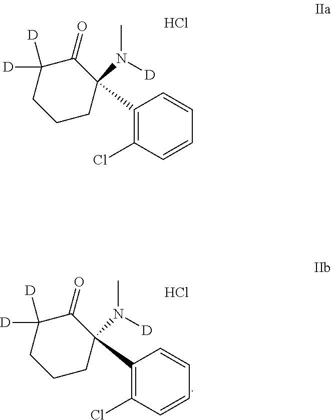

In preferred embodiments, the compound of Formula Ia or Ib is a deuterium chloride salt of d2-R-ketamine, d2-S-ketamine, or mixtures thereof:

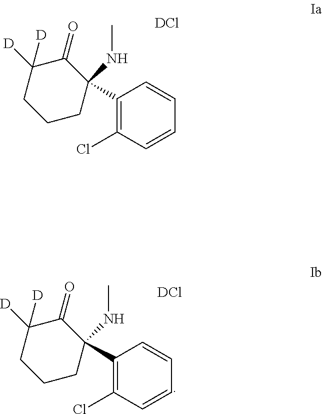

In other preferred embodiments, the compound of Formula IIa or IIb is a deuterium chloride salt of d3-R-ketamine, d3-S-ketamine or mixtures thereof:

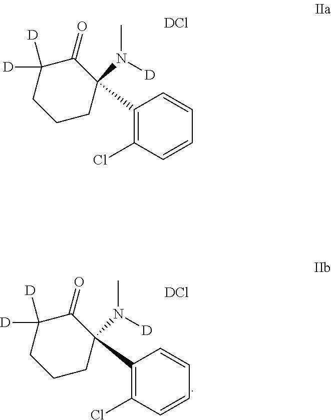

As discussed above, the compound of Formula I and/or II provides a deuterium substituted ketamine. In some embodiments, each deuterium of the compound of Formula I and/or II independently has deuterium enrichment of no less than about 1%, no less than about 5%, no less than about 10%, no less than about 20%, no less than about 50%, no less than about 70%, no less than about 80%, no less than about 90%, or no less than about 98%. In other embodiments, both deuteriums in the compound of Formula I have deuterium enrichment of no less than about 1% or 10%. In other embodiments, two or three deuteriums in the compound of Formula II have deuterium enrichment of no less than about 1 or 10%. In some embodiments, the compositions disclosed herein comprise the compound of Formula I and/or II as a single enantiomer. In other embodiments, the compounds and compositions are racemic comprising a mixture of the enantiomers. For example, in some aspects, compositions comprise about 90% or more by weight of the (R) enantiomer. In other aspects, compositions comprise about 80% by weight of the (R) enantiomer. In other aspects, compositions comprise about 70% by weight of the (R) enantiomer. In other aspects, compositions comprise about 60% by weight of the (R) enantiomer. In other aspects, compositions comprise about 50% by weight of the (R) enantiomer. In other aspects, compositions comprise about 40% by weight of the (R) enantiomer. In other aspects, compositions comprise about 30% by weight of the (R) enantiomer. In other aspects, compositions comprise about 20% by weight of the (R) enantiomer. In other aspects, compositions comprise about 10% by weight of the (R) enantiomer. In other aspects, compositions comprise about 5% by weight of the (R) enantiomer.

In some aspects, compositions comprise about 90% or more by weight of the (S) enantiomer. In other aspects, compositions comprise about 80% by weight of the (S) enantiomer. In other aspects, compositions comprise about 70% by weight of the (S) enantiomer. In other aspects, compositions comprise about 60% by weight of the (S) enantiomer. In other aspects, compositions comprise about 50% by weight of the (S) enantiomer. In other aspects, compositions comprise about 40% by weight of the (S) enantiomer. In other aspects, compositions comprise about 30% by weight of the (S) enantiomer. In other aspects, compositions comprise about 20% by weight of the (S) enantiomer. In other aspects, compositions comprise about 10% by weight of the (S) enantiomer. In other aspects, compositions comprise about 5% by weight of the (S) enantiomer.

In certain embodiments, the compound of Formula I and/or II as disclosed herein contains about 60% or more by weight of the (S)-enantiomer of the compound and about 40% or less by weight of (R)-enantiomer of the compound. In certain embodiments, the compound of Formula I and/or II as disclosed herein contains about 70% or more by weight of the (S)-enantiomer of the compound and about 30% or less by weight of (R)-enantiomer of the compound. In certain embodiments, the compound of Formula I and/or II as disclosed herein contains about 80% or more by weight of the (S)-enantiomer of the compound and about 20% or less by weight of (R)-enantiomer of the compound. In certain embodiments, the compound of Formula I and/or II as disclosed herein contains about 90% or more by weight of the (S)-enantiomer of the compound and about 10% or less by weight of the (R)-enantiomer of the compound. In certain embodiments, the compound of Formula I and/or II as disclosed herein contains about 95% or more by weight of the (S)-enantiomer of the compound and about 5% or less by weight of (R)-enantiomer of the compound. In certain embodiments, the compound of Formula I and/or II as disclosed herein contains about 99% or more by weight of the (S)-enantiomer of the compound and about 1% or less by weight of (R)-enantiomer of the compound.

In certain embodiments, the compound of Formula I and/or II as disclosed herein contains about 60% or more by weight of the (R)-enantiomer of the compound and about 40% or less by weight of (S)-enantiomer of the compound. In certain embodiments, the compound of Formula I and/or II as disclosed herein contains about 70% or more by weight of the (R)-enantiomer of the compound and about 30% or less by weight of (S)-enantiomer of the compound. In certain embodiments, the compound of Formula I and/or II as disclosed herein contains about 80% or more by weight of the (R)-enantiomer of the compound and about 20% or less by weight of (S)-enantiomer of the compound. In certain embodiments, the compound of Formula I and/or II as disclosed herein contains about 90% or more by weight of the (R)-enantiomer of the compound and about 10% or less by weight of the (S)-enantiomer of the compound. In certain embodiments, the compound of Formula I and/or II as disclosed herein contains about 95% or more by weight of the (R)-enantiomer of the compound and about 5% or less by weight of (S)-enantiomer of the compound. In certain embodiments, the compound of Formula I and/or II as disclosed herein contains about 99% or more by weight of the (R)-enantiomer of the compound and about 1% or less by weight of (S)-enantiomer of the compound.

The compound of Formula I and/or II as disclosed herein may also contain less prevalent isotopes for other elements, including, but not limited to, ¹³C or ¹⁴C for carbon, ³³S³⁴S, or ³⁶S for sulfur, ¹⁵N for nitrogen, and ¹⁷O or ¹⁸O for oxygen.

In certain embodiments, without being bound by any theory, the compounds disclosed herein, including compounds of Formula I and/or II, may expose a patient to a maximum of about 0.000005% D₂O or about 0.00001% DHO, assuming that all of the C-D bonds in the compound as disclosed herein are metabolized and released as D₂O or DHO. This quantity is a small fraction of the naturally occurring background levels of D₂O or DHO in circulation. In certain embodiments, the levels of D₂O shown to cause toxicity in animals is much greater than even the maximum limit of exposure because of the deuterium enriched compound as disclosed herein. Thus, in certain embodiments, the deuterium-enriched compound disclosed herein should not cause any additional toxicity because of the use of deuterium.

In some embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), modifying AUC, lowering the efficacious dose and thus decreasing the non-mechanism-related toxicity, and/or lowering the probability of drug-drug interactions.

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

Further provided are processes for preparing a compound as disclosed herein as a NMDA receptor modulator, or other pharmaceutically acceptable derivatives such as prodrug derivatives, or individual isomers and mixture of isomers or enantiomers thereof. The compounds as disclosed herein can be prepared by methods known to one of skill in the art and routine modifications thereof, and/or following procedures similar to those described in the Examples section herein and routine modifications thereof, and/or procedures found in Hopfgartner et al., J. Mass. Spectrom. 1996, 31, 69-76, U.S. Pat. No. 3,254,124, and references cited therein and routine modifications thereof. Compounds as disclosed herein can also be prepared as shown in any of the following schemes and routine modifications thereof. For example, certain compounds as disclosed herein can be prepared as shown in Example 1 hereinbelow.

Pharmaceutical Compositions

Disclosed herein are pharmaceutical compositions comprising a compound as disclosed herein as an active ingredient, or a pharmaceutically acceptable salt, solvate, or prodrug thereof, in a pharmaceutically acceptable vehicle, carrier, diluent, or excipient, or a mixture thereof; in combination with one or more pharmaceutically acceptable excipients or carriers. Pharmaceutical compositions prepared using D2-esketamine or its salts (as described herein) have improved stability, as compared to pharmaceutical compositions including esketamine or other deuterated derivatives, such as D6-esketamine and D8-esketamine, as the active ingredient. The improved stability of the D2-esketamine-containing pharmaceutical compositions could not have been expected.

The pharmaceutical compositions that comprise a compound disclosed herein may be formulated in various dosage forms for oral, intranasal, parenteral, or topical administration. The pharmaceutical compositions may also be formulated as an immediate or modified release dosage form, including delayed-, extended-, prolonged-, sustained-, pulsatile-, controlled-, accelerated- and fast-, targeted-, programmed-release, and gastric retention dosage forms, and may be optionally coated. These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).

The pharmaceutical compositions disclosed herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. In some embodiments, the pharmaceutical composition comprises a tablet or capsule. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles or packages comprising tablets or capsules, or bottles of pints or gallons.

The compound as disclosed herein may be administered alone, or in combination with one or more other compounds disclosed herein, one or more other active ingredients.

The pharmaceutical compositions disclosed herein may be administered as single or multiple doses at intervals of time.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose may be administered. Subsequently, the dosage or the frequency of administration, or both, can be modified, as a function of the symptoms, to a level at which the improved disease, disorder or condition is retained. Patients can, however, require intermittent treatment on a long-term basis upon any recurrence of symptoms.

A. Oral Administration

The pharmaceutical compositions disclosed herein may be formulated in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions disclosed herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions disclosed herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrrolidine. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions disclosed herein may be formulated as compressed tablets, tablet triturates, chewable lozenges, rapidly dissolving tablets, multiple compressed tablets, or coated forms such as enteric-coated tablets, sugar-coated, or film-coated tablets.

In some embodiments, the oral dosage form is coated. In some embodiments, the oral dosage form is coated with an enteric coating. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets. In some embodiments, disclosed are pharmaceutical compositions in enteric coated dosage forms, which comprise a compound as disclosed herein, or a pharmaceutically acceptable salt, solvate, or prodrug thereof; and one or more release controlling excipients or carriers for use in an enteric coated dosage form. The pharmaceutical compositions may also comprise non-release controlling excipients or carriers.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions disclosed herein may be formulated as soft or hard capsules, which can be made from gelatin, methylcellulose, starch, or calcium alginate. The hard gelatin capsule, also known as the dry-filled capsule, consists of two sections, one slipping over the other, thus completely enclosing the active ingredient. The soft elastic capsule is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions disclosed herein may be formulated in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene, butylated hydroxyanisole, propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions disclosed herein for oral administration may be also formulated in the forms of liposomes, micelles, microspheres, or nanosystems. Micellar dosage forms can be prepared as described in, e.g., U.S. Pat. No. 6,350,458.

The pharmaceutical compositions disclosed herein may be formulated as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide. In some embodiments, the pharmaceutical compositions are provided in an effervescent dosage forms, which comprise a compound as disclosed herein, or a pharmaceutically acceptable salt, solvate, or prodrug thereof; and one or more release controlling excipients or carriers for use in an effervescent dosage form. The pharmaceutical compositions may also comprise non-release controlling excipients or carriers.

Coloring and flavoring agents can be used in all of the above dosage forms.

The pharmaceutical compositions disclosed herein may be formulated as immediate or modified release dosage forms, including delayed-, sustained, pulsed-, controlled, targeted-, and programmed-release forms.

The pharmaceutical compositions disclosed herein may be co-formulated with other active ingredients which do not impair the desired therapeutic action, or with substances that supplement the desired action, such as drotrecogin-α, and hydrocortisone.

In other embodiments, pharmaceutical compositions in a dosage form for oral administration are provided. Such compositions comprise a compound as disclosed herein, or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In further embodiments, the pharmaceutical compositions comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg of one or more compounds as disclosed herein. In some embodiments, the compounds are formulated as enteric-coated granules, as delayed-release capsules for oral administration. In some embodiments, the pharmaceutical composition further comprises cellulose, disodium hydrogen phosphate, hydroxypropyl cellulose, hypromellose, lactose, mannitol, and sodium lauryl sulfate. In other embodiments, the pharmaceutical composition further comprises glyceryl monostearate 40-50, hydroxypropyl cellulose, hypromellose, magnesium stearate, methacrylic acid copolymer type C, polysorbate 80, sugar spheres, talc, and triethyl citrate.

In some embodiments, the pharmaceutical composition further comprises carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide. In other embodiments, the pharmaceutical composition further comprises calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

B. Parenteral Administration

The pharmaceutical compositions disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are formulated as ready-to-use sterile solutions. In another embodiment, the pharmaceutical compositions are formulated as sterile dry soluble products, including lyophilized powders and hypodermic tablets, to be reconstituted with a vehicle prior to use. In yet another embodiment, the pharmaceutical compositions are formulated as ready-to-use sterile suspensions. In yet another embodiment, the pharmaceutical compositions are formulated as sterile dry insoluble products to be reconstituted with a vehicle prior to use. In still another embodiment, the pharmaceutical compositions are formulated as ready-to-use sterile emulsions.

The pharmaceutical compositions disclosed herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions disclosed herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

C. Topical Administration

The pharmaceutical compositions disclosed herein may be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may also comprise liposomes, micelles, microspheres, nanosystems, and mixtures thereof.

Pharmaceutically acceptable carriers and excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions disclosed herein may be formulated in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions disclosed herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

The pharmaceutical compositions disclosed herein may be administered intranasally or by inhalation to the respiratory tract. The pharmaceutical compositions may be formulated in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be formulated as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient disclosed herein, a propellant as solvent; and/or an surfactant, such as sorbitan trioleate, oleic acid, or an oligolactic acid.

The pharmaceutical compositions disclosed herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions disclosed herein; a suitable powder base, such as lactose or starch; and a performance modifier, such as l-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions disclosed herein for inhalation or /intranasal administration may further comprise a suitable flavor, such as menthol and levomenthol, or sweeteners, such as saccharin or saccharin sodium.

The pharmaceutical compositions disclosed herein for topical administration may be formulated to be immediate release or modified release, including delayed-, sustained-, pulsed-, controlled-, targeted, and programmed release.

D. Modified Release

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. In some embodiments, the pharmaceutical composition comprises a compound as disclosed herein, or a pharmaceutically acceptable salt, solvate, or prodrug thereof; and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. Thus, in certain embodiments, the pharmaceutical composition comprises one or more release-controlling excipients. The pharmaceutical compositions may also comprise non-release controlling excipients or carriers. In some embodiments, the pharmaceutical composition further comprises one or more non-release controlling excipients.

The pharmaceutical compositions disclosed herein may be formulated as an abuse deterrent dosage form. Examples of modified release include, but are not limited to, those described in US20170035707, WO2015151259, US20150118302; US20150118303, US20160250203, US20160256392, US20160317457.

The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

1. Matrix Controlled Release Devices

The pharmaceutical compositions disclosed herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, and the particle sizes of the polymer and/or the active ingredient, the ratio of the active ingredient versus the polymer, and other excipients or carriers in the compositions. The pharmaceutical compositions disclosed herein in a modified release dosage form may be prepared by methods known to those skilled in the art, including direct compression, dry or wet granulation followed by compression, melt-granulation followed by compression.

2. Osmotic Controlled Release Devices

The pharmaceutical compositions disclosed herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s) and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), PEG, polypropylene glycol (PPG), poly(2-hydroxyethyl methacrylate), poly(acrylic) acid, poly(methacrylic) acid, PVP, crosslinked PVP, PVA, PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, HEC, HPC, HPMC, CMC and carboxyethyl, cellulose, sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced CA, cellulose diacetate, cellulose triacetate, CA propionate, cellulose nitrate, CAB, CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions disclosed herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and International Patent Publication No. WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions disclosed herein are formulated as ESC controlled-release dosage form, which comprises an osmotic membrane that coats a core comprising the active ingredient(s), a hydroxylethyl cellulose, and other pharmaceutically acceptable excipients or carriers.

3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

4. Targeted Delivery

The pharmaceutical compositions disclosed herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

5. Immediate Release Delivery

The compounds or compositions discussed herein may be formulated for delivery to a subject by immediate-release. In some embodiments, the compounds or compositions are formulated as discussed in U.S. Patent Publication No. 2016-0317457.

6. Combined Release Delivery

Additionally disclosed are pharmaceutical compositions in a dosage form that has an instant releasing component and at least one delayed releasing component, and is capable of giving a discontinuous release of the compound in the form of at least two consecutive pulses separated in time from 0.1 up to 24 hours. The pharmaceutical compositions comprise a compound as disclosed herein, or a pharmaceutically acceptable salt, solvate, or prodrug thereof; and one or more release controlling and non-release controlling excipients or carriers, such as those excipients or carriers suitable for a disruptable semi-permeable membrane and as swellable substances.

Methods of Use

Any one of the compounds of formula I, Ia, Ib, II, Ia and IIb disclosed herein are useful in inducing a response in a subject, where a similar response is achieved when using ketamine. Accordingly, disclosed are methods for treating, preventing, or ameliorating one or more symptoms of a ketamine responsive disorder, comprising administering to a subject having or being suspected to have such a disorder, a therapeutically effective amount of a compound as disclosed herein; or a pharmaceutically acceptable salt, solvate, or prodrug thereof. In some embodiments, the ketamine responsive disorder can be lessened, alleviated, or prevented by using an agent which is an anesthetic, analgesic, entheogen, therapeutic cataleptic, and neuroprotectant. Preferably, the anesthetic promotes general anesthesia.

It has been determined that various receptors can be modulated by ketamine. Thus, also within the scope of the disclosure, are methods of modulating the activity of one or more type of receptors that are modulated by ketamine. Also disclosed herein are methods of modulating the activity of receptors that respond to the action of ketamine. In some embodiments, these methods include contacting the receptors with at least one compound as disclosed herein; or a pharmaceutically acceptable salt, solvate, or prodrug thereof.

Ketamine responsive disorders include, but are not limited to, to alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, pain, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder and the like. In some embodiments, the disorder is Rett syndrome. In some embodiments the disorder is depression, more preferably, major depressive disorder, refractory depression, treatment resistant depression, or depression associated with a genetic disorder.

Examples of receptors that are modulated by ketamine are the NMDA receptors and the AMPA receptors. In some embodiments, disclosed are methods for treating, preventing, or ameliorating one or more symptoms of an NMDA receptor mediated-disorder, comprising administering to a subject having or being suspected to have such a disorder, a therapeutically effective amount of a compound as disclosed herein; or a pharmaceutically acceptable salt, solvate, or prodrug thereof. In other embodiments, disclosed are methods for treating, preventing, or ameliorating one or more symptoms of an AMPA receptor mediated-disorder, comprising administering to a subject having or being suspected to have such a disorder, a therapeutically effective amount of a compound as disclosed herein; or a pharmaceutically acceptable salt, solvate, or prodrug thereof.

NMDA receptor mediated-disorders include, but are not limited to, alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, pain, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder. In some embodiments, NDMA receptor-mediated disorder is nociceptive pain, neuropathic pain, phantom limb pain, ischemic pain, stroke, sepsis, inflammation, opioid tolerance, Alzheimer's disease, or burn. In some embodiments, the disorder is depression and, preferably, major depressive disorder, refractory depression, treatment resistant depression, or depression associated with a genetic disorder.

Also disclosed are methods of treating, preventing, or ameliorating one or more symptoms of a disorder associated with NMDA receptors, by administering to a subject having or being suspected to have such a disorder, a therapeutically effective amount of a compound as disclosed herein; or a pharmaceutically acceptable salt, solvate, or prodrug thereof.

Further disclosed are methods of treating, preventing, or ameliorating one or more symptoms of a disorder responsive to modulation of NMDA receptors, comprising administering to a subject having or being suspected to have such a disorder, a therapeutically effective amount of a compound as disclosed herein; or a pharmaceutically acceptable salt, solvate, or prodrug thereof.

In some embodiments, the NMDA receptor-mediated disorder can be lessened, alleviated, or prevented by using an agent which is an anesthetic, analgesic, entheogen, therapeutic cataleptic, and neuroprotectant. Preferably, the anesthetic promotes general anesthesia.

Furthermore, disclosed herein are methods of modulating the activity of NMDA receptors, comprising contacting the receptors with at least one compound as disclosed herein; or a pharmaceutically acceptable salt, solvate, or prodrug thereof. In one embodiment, the NMDA receptor(s) are expressed by a cell. Also disclosed herein are methods of modulating the activity of AMPA receptors, comprising contacting the receptors with at least one compound as disclosed herein; or a pharmaceutically acceptable salt, solvate, or prodrug thereof. In one embodiment, the AMPA receptor(s) are expressed by a cell.

Disclosed herein are methods for treating a subject, including a human, having or suspected of having a disorder involving, but not limited to, alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, pain, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder, or for preventing such a disorder in a subject prone to the disorder; comprising administering to the subject a therapeutically effective amount of a compound as disclosed herein; or a pharmaceutically acceptable salt, solvate, or prodrug thereof; so as to affect decreased inter-individual variation in plasma levels of the compound or a metabolite thereof, during the treatment of the disorder as compared to the corresponding non-isotopically enriched compound.

In certain embodiments, the inter-individual variation in plasma levels of the compounds as disclosed herein, or metabolites thereof, is decreased by greater than about 5%, greater than about 10%, greater than about 15%, greater than about 20%, greater than about 25%, greater than about 30%, greater than about 35%, greater than about 40%, greater than about 45%, or by greater than about 50% as compared to the corresponding non-isotopically enriched compound.

Disclosed herein are methods for treating a subject, including a human, having or suspected of having a disorder involving, but not limited to, alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, pain, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder, or for preventing such a disorder in a subject prone to the disorder; comprising administering to the subject a therapeutically effective amount of a compound as disclosed herein; or a pharmaceutically acceptable salt, solvate, or prodrug thereof; so as to affect increased average plasma levels of the compound or decreased average plasma levels of at least one metabolite of the compound per dosage unit as compared to the corresponding non-isotopically enriched compound.

In certain embodiments, the average plasma levels of the compound as disclosed herein are increased by greater than about 5%, greater than about 10%, greater than about 1510%, greater than about 20%, greater than about 25%, greater than about 30%, greater than about 35%, greater than about 40%, greater than about 45%, or greater than about 50% as compared to the corresponding non-isotopically enriched compounds.

In certain embodiments, the average plasma levels of a metabolite of the compound as disclosed herein are decreased by greater than about 5%, greater than about 10%, greater than about 15%, greater than about 20%, greater than about 25%, greater than about 30%, greater than about 35%, greater than about 40%, greater than about 45%, or greater than about 50% as compared to the corresponding non-isotopically enriched compounds

Plasma levels of the compound as disclosed herein, or metabolites thereof, are measured using the methods described by Li et al. (Rapid Communications in Mass Spectrometry 2005, 19, 1943-1950).

Disclosed herein are methods for treating a subject, including a human, having or suspected of having a ketamine or ketamine metabolite responsive disorder involving, but not limited to, alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, pain, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder, or for preventing such a disorder in a subject prone to the disorder; comprising administering to the subject a therapeutically effective amount of a compound as disclosed herein, or a pharmaceutically acceptable salt, solvate, or prodrug thereof; so as to affect a decreased inhibition of, and/or metabolism by at least one cytochrome P₄₅₀ or monoamine oxidase isoform in the subject during the treatment of the disease as compared to the corresponding non-isotopically enriched compound.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, or CYP51.Examples of monoamine oxidase isoforms in a mammalian subject include, but are not limited to, MAO_{A}, and MAO_{B}.

In certain embodiments, the decrease in inhibition of the cytochrome P₄₅₀ or monoamine oxidase isoform by a compound as disclosed herein is greater than about 5%, greater than about 10%, greater than about 15%, greater than about 20%, greater than about 25%, greater than about 30%, greater than about 35%, greater than about 40%, greater than about 45%, or greater than about 50% as compared to the corresponding non-isotopically enriched compounds.

The inhibition of the cytochrome P₄₅₀ isoform is measured by the method of Ko et al. (British Journal of Clinical Pharmacology, 2000, 49, 343-351). The inhibition of the MAO_{A} isoform is measured by the method of Weyler et al. (J. Biol. Chem. 1985, 260, 13199-13207). The inhibition of the MAO_{B} isoform is measured by the method of Uebelhack et al. (Pharmacopsychiatry, 1998, 31, 187-192).

Disclosed herein are methods for treating a subject, including a human, having or suspected of having a disorder involving, but not limited to, alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, pain, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder, or for preventing such a disorder in a subject prone to the disorder; comprising administering to the subject a therapeutically effective amount of a compound as disclosed herein, or a pharmaceutically acceptable salt, solvate, or prodrug thereof; so as to affect a decreased metabolism via at least one polymorphically-expressed cytochrome P₄₅₀ isoform in the subject during the treatment of the disorder as compared to the corresponding non-isotopically enriched compound. In other embodiments, the compound has an increased or decreased metabolism by at least one polymorphically-expressed cytochrome P₄₅₀ isoform in the subject per dosage unit thereof as compared to the non-isotopically enriched compound. In further embodiments, the compound is characterized by increased or decreased inhibition of at least one cytochrome P₄₅₀ or monoamine oxidase isoform in said subject per dosage unit thereof as compared to the non-isotopically enriched compound.

Examples of polymorphically-expressed cytochrome P₄₅₀ isoforms in a mammalian subject include, but are not limited to, CYP2C8, CYP2C9, CYP2C19, and CYP2D6.

In certain embodiments, the decrease in metabolism of the compound as disclosed herein by at least one polymorphically-expressed cytochrome P₄₅₀ isoforms cytochrome P₄₅₀ isoform is greater than about 5%, greater than about 10%, greater than about 15%, greater than about 20%, greater than about 25%, greater than about 30%, greater than about 35%, greater than about 40%, greater than about 45%, or greater than about 50% as compared to the corresponding non-isotopically enriched compound.

The metabolic activities of the cytochrome P₄₅₀ isoforms are measured, for example, by the method described in Example 4. The metabolic activities of the monoamine oxidase isoforms are measured, for example, by the methods described in Examples 5, and 6.

Disclosed herein are methods for treating a subject, including a human, having or suspected of having a disorder involving, but not limited to, alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, pain, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder, or for preventing such a disorder in a subject prone to the disorder; comprising administering to the subject a therapeutically effective amount of a compound as disclosed herein, or a pharmaceutically acceptable salt, solvate, or prodrug thereof; so as to affect at least one statistically-significantly improved disorder-control and/or disorder-eradication endpoint, as compared to the corresponding non-isotopically enriched compound. Examples of improved disorder-control and/or disorder-eradication endpoints include, but are not limited to, statistically-significant improvement of pain indices, perfusion of ischemic tissues with oxygen, prevention of ischemia, entheogenic effects sufficient to facilitate psychotherapy, cataleptic effects sufficient to enable medical treatment of a non-compliant trauma victim, neuroprotection during an ischemic event, and/or diminution of hepatotoxicity, as compared to the corresponding non-isotopically enriched compound.

Disclosed herein are methods for treating a subject, including a human, having or suspected of having a disorder involving, but not limited to, alcohol dependence, Alzheimer's disease, anxiety, asthma spectrum disorder, autism, bipolar disorder, Bulbar function depression, burn, diabetic neuropathy, dyskinesia, epilepsy fibromyalgia, ischemic pain, inflammation, obsessive-compulsive disorder, pain, major depressive disorder, nociceptive pain, neuropathic pain, opioid tolerance, phantom limb, post-traumatic stress syndrome, pseudobulbar effect, Rett syndrome, refractory depression, schizophrenia, sepsis, stroke, suicidality, traumatic brain injury, treatment resistant depression, tinnitus, and depression associated with genetic disorders and the like, or for preventing such a disorder in a subject prone to the disorder; comprising administering to the subject a therapeutically effective amount of a compound as disclosed herein, or a pharmaceutically acceptable salt, solvate, or prodrug thereof; so as to affect an improved clinical effect as compared to the corresponding non-isotopically enriched compound. Examples of improved clinical effects include, but are not limited to, statistically-significant improvement of pain or depression indices, perfusion of ischemic tissues with oxygen, prevention of ischemia, entheogenic effects sufficient to facilitate psychotherapy, cataleptic effects sufficient to enable medical treatment of a non-compliant trauma victim, improvement in cognition, neuroprotection during an ischemic event, and/or diminution of hepatotoxicity, or any relevant safety measures as compared to the corresponding non-isotopically enriched compound.

Disclosed herein are methods for treating a subject, including a human, having or suspected of having a disorder involving, but not limited to, alcohol dependence, Alzheimer's disease, anxiety, asthma spectrum disorder, autism, bipolar disorder, Bulbar function depression, burn, diabetic neuropathy, dyskinesia, epilepsy fibromyalgia, ischemic pain, inflammation, obsessive-compulsive disorder, pain, major depressive disorder, nociceptive pain, neuropathic pain, opioid tolerance, phantom limb, post-traumatic stress syndrome, pseudobulbar effect, Rett syndrome, refractory depression, schizophrenia, sepsis, stroke, suicidality, traumatic brain injury, treatment resistant depression, tinnitus, and depression associated with genetic disorders and the like., or for preventing such a disorder in a subject prone to the disorder; comprising administering to the subject a therapeutically effective amount of a compound as disclosed herein, or a pharmaceutically acceptable salt, solvate, or prodrug thereof; so as to affect prevention of recurrence, or delay of decline or appearance, of abnormal alimentary or hepatic parameters as the primary clinical benefit, as compared to the corresponding non-isotopically enriched compound.

Disclosed herein are methods for treating a subject, including a human, having or suspected of having a disorder involving, but not limited to, alcohol dependence, Alzheimer's disease, anxiety, asthma spectrum disorder, autism, bipolar disorder, Bulbar function depression, burn, diabetic neuropathy, dyskinesia, epilepsy fibromyalgia, ischemic pain, inflammation, obsessive-compulsive disorder, pain, major depressive disorder, nociceptive pain, neuropathic pain, opioid tolerance, phantom limb, post-traumatic stress syndrome, pseudobulbar effect, Rett syndrome, refractory depression, schizophrenia, sepsis, stroke, suicidality, traumatic brain injury, treatment resistant depression, tinnitus, and depression associated with genetic disorders and the like. or for preventing such a disorder in a subject prone to the disorder; comprising administering to the subject a therapeutically effective amount of a compound as disclosed herein, or a pharmaceutically acceptable salt, solvate, or prodrug thereof; so as to allow the treatment of, but not limited to, alcohol dependence, Alzheimer's disease, anxiety, asthma spectrum disorder, autism, bipolar disorder, Bulbar function depression, burn, diabetic neuropathy, dyskinesia, epilepsy fibromyalgia, ischemic pain, inflammation, obsessive-compulsive disorder, pain, major depressive disorder, nociceptive pain, neuropathic pain, opioid tolerance, phantom limb, post-traumatic stress syndrome, pseudobulbar effect, Rett syndrome, refractory depression, schizophrenia, sepsis, stroke, suicidality, traumatic brain injury, treatment resistant depression, tinnitus, and depression associated with genetic disorders and the like, while reducing or eliminating deleterious changes in abnormal alimentary, hepatic parameter, or diagnostic hepatobiliary function endpoints as compared to the corresponding non-isotopically enriched compound. In some embodiments, the method affects treatment of the disorder while reducing or eliminating a deleterious change in a diagnostic hepatobiliary function endpoint, as compared to the corresponding non-isotopically enriched compound. In some embodiments, the method affects treatment of the disorder while reducing or eliminating an abnormal alimentary or hepatic parameter, as compared to the corresponding non-isotopically enriched compound. Examples of diagnostic hepatobiliary function endpoints include, but are not limited to, alanine aminotransferase (ALT), serum glutamic-pyruvic transaminase (SGPT), aspartate aminotransferase (AST or SGOT), ALT/AST ratios, serum aldolase, alkaline phosphatase (ALP), ammonia levels, bilirubin, gamma-glutamyl transpeptidase (GGTP, γ-GTP, or GGT), leucine aminopeptidase (LAP), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

In some embodiments, the compound has at least one of the following properties: a) decreased inter-individual variation in plasma levels of said compound or a metabolite thereof as compared to the non-isotopically enriched compound; b) increased average plasma levels of said compound per dosage unit thereof as compared to the non-isotopically enriched compound; c) decreased average plasma levels of at least one metabolite of said compound per dosage unit thereof as compared to the non-isotopically enriched compound; d) increased average plasma levels of at least one metabolite of said compound per dosage unit thereof as compared to the non-isotopically enriched compound; or e) an improved clinical effect during the treatment in said subject per dosage unit thereof as compared to the non-isotopically enriched compound.

In yet further embodiments, the compound has at least two of the following properties: a) decreased inter-individual variation in plasma levels of said compound or a metabolite thereof as compared to the non-isotopically enriched compound; b) increased average plasma levels of said compound per dosage unit thereof as compared to the non-isotopically enriched compound; c) decreased average plasma levels of at least one metabolite of said compound per dosage unit thereof as compared to the non-isotopically enriched compound; d) increased average plasma levels of at least one metabolite of said compound per dosage unit thereof as compared to the non-isotopically enriched compound; and e) an improved clinical effect during the treatment in said subject per dosage unit thereof as compared to the non-isotopically enriched compound.

Also provided are methods directed to decreasing the production of metabolites of ketamine, for example, decreasing the production of inactive metabolites of ketamine. In some embodiments, the disclosure provides methods of decreasing the production of hydroxynorketamine in a subject. In other embodiments, the subject had previously been administered ketamine. In further embodiments, the subject had not previously been administered ketamine. Such methods comprise administering to the subject a compound or pharmaceutical composition disclosed herein.

Further provided are methods of increasing the production of metabolites of ketamine, for example, increasing the producing of active metabolites of ketamine. In some embodiments, the disclosure provides methods of increasing the production of norketamine in a subject. In other embodiments, the subject had been previously administered ketamine. In further embodiments, the subject has not previously been administered ketamine. The methods comprise administering to the subject a compound or pharmaceutical composition described herein.

Depending on the disease to be treated and the subject's condition, the compound as disclosed herein disclosed herein may be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration, and may be formulated, alone or together, in suitable dosage unit with pharmaceutically acceptable carriers, adjuvants and vehicles appropriate for each route of administration.

The dose may be in the form of one, two, three, four, five, six, or more sub-doses that are administered at appropriate intervals per day or per week or per month. The dose or sub-doses can be administered in the form of dosage units containing from about 0.1 to about 1000 milligram, from about 0.1 to about 500 milligrams, or from 0.5 about to about 100 milligram active ingredient(s) per dosage unit, and if the condition of the patient requires, the dose can, by way of alternative, be administered as a continuous infusion. In other embodiments, the compounds are administered in a dose of about 0.5 milligram to about 1000 milligrams. Preferably, the compounds are administered in a dose of about 1 to about 100 milligrams, about 1 to about 50 milligrams, about 5 to about 20 milligrams, or about 50 to about 100milligrams.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

Combination Therapy

The compounds disclosed herein may also be combined or used in combination with other agents useful in the treatment, prevention, or amelioration of one or more symptoms of the disorders for which the compound disclosed herein are useful, including, but not limited to, alcohol dependence, Alzheimer's disease, anxiety, asthma spectrum disorder, autism, bipolar disorder, Bulbar function depression, burn, diabetic neuropathy, dyskinesia, epilepsy, fibromyalgia, ischemic pain, inflammation, obsessive-compulsive disorder, pain, major depressive disorder, pain such as nociceptive pain or neuropathic pain, opioid tolerance, phantom limb, post-traumatic stress syndrome, pseudobulbar effect, Rett syndrome, refractory depression, schizophrenia, sepsis, stroke, suicidality, tinnitus, traumatic brain injury, treatment resistant depression, or depression associated with a genetic disorder. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). The compositions and methods disclosed herein may be used as monotherapy or as adjunct therapy. Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required. Accordingly, the pharmaceutical compositions disclosed herein include those that also contain one or more other active ingredients or therapeutic agents, in addition to the compound disclosed herein.

The compounds, compositions and or methods pharmaceutical compositions may further comprise another therapeutic agent for combination therapy. In some embodiments, the therapeutic agent is a NMDA-receptor modulator, opioid, anesthetic, peripherally acting muscle relaxant, benzodiazepine, endothelin converting enzyme (ECE) inhibitor, thromboxane enzyme antagonist, potassium channel opener, thrombin inhibitor, growth factor inhibitor, platelet activating factor (PAF) antagonist, anti-platelet agent, Factor VIIa inhibitor, Factor Xa inhibitor, renin inhibitor, neutral endopeptidase (NEP) inhibitor, vasopepsidase inhibitor, HMG CoA reductase inhibitor, squalene synthetase inhibitor, fibrate, bile acid sequestrant, anti-atherosclerotic agent, MTP inhibitor, calcium channel blocker, potassium channel activator, alpha-PDE5 agent, beta-PDE5 agent, antiarrhythmic agent, diuretic, anti-diabetic agent, PPAR-gamma agonist, mineralocorticoid enzyme antagonist, aP2 inhibitor, protein tyrosine kinase inhibitor, antiinflammatory, antiproliferative, chemotherapeutic agent, immunosuppressant, anticancer agent, cytotoxic agent, antimetabolite, farnesyl-protein transferase inhibitor, hormonal agent, microtubule-disruptor agent, microtubule-stabilizing agent, topoisomerase inhibitor, prenyl-protein transferase inhibitor, cyclosporin, TNF-alpha inhibitor, cyclooxygenase-2 (COX-2) inhibitor, gold compound, or platinum coordination complex.

In certain embodiments, the compounds, compositions and or methods disclosed herein can be combined with one or more modulators of NMDA-receptors known in the art, including, but not limited to, phencyclidine (PCP), amantadine, ibogaine, memantine, nitrous oxide, and dextromethorphan. In some embodiments, the NMDA receptor-mediated disorder can be lessened, alleviated, or prevented by administering a NDMA receptor modulator. In other embodiments, the ketamine responsive disorder can be lessened, alleviated, or prevented by administering a NDMA receptor modulator.

In certain embodiments, the compounds, compositions and or methods disclosed herein can be combined with one or more natural, semisynthetic, or fully synthetic opioids known in the art, including, but not limited to, morphine, codeine, thebain, diacetylmorphine, oxycodone, hydrocodone, hydromorphone, oxymorphone, nicomorphine, fentanyl, α-methylfentanyl, alfentanil, sufentanil, remifentanyl, carfentanyl, ohmefentanyl, pethidine, ketobemidone, propoxyphene, dextropropoxyphene, methadone, loperamide, pentazocine, buprenorphine, etorphine, butorphanol, nalbufine, levorphanol, naloxone, naltrexone, and tramadol.

In certain embodiments, the compounds, compositions and or methods provided herein can be combined with one or more local or general anesthetics known in the art, including, but not limited to, diethyl ether, vinyl ether, halothane, chloroform, methoxyflurane, enflurane, trichloroethylene, isoflurane, desflurane, sevoflurane, methohexital, hexobarbital, thiopental, narcobarbital, fentanyl, alfentanil, sufentanil, phenoperidine, anileridine, remifentanil, droperidol, non-deuterated ketamine, propanidid, alfaxalone, etomidate, propofol, hydroxybutyric acid, nitrous oxide, non-deuterated esketamine, metabutethamine, procaine, tetracaine, chloroprocaine, benzocaine, bupivacaine, lidocaine, mepivacaine, prilocaine, butanilicaine, cinchocaine, etidocaine, articaine, ropivacaine, levobupivacaine, cocaine, ethyl chloride, dyclonine, phenol, and capsaicin.

In certain embodiments, the compounds, compositions and or methods disclosed herein can be combined with one or more peripherally acting muscle relaxants known in the art, including, but not limited to alcuronium, dimethyltubocurarine, tubocurarine, suxamethonium, atracurium, cisatracurium, doxacurium chloride, fazadinium bromide, gallamine, hexafluronium, mivacurium chloride, pancuronium, pipecuronium bromide, rocuronium bromide, vecuronium, and botulinum toxin.

In certain embodiments, the compounds, compositions and or methods disclosed herein can be combined with one or more benzodiazepines (“minor tranquilizers”) known in the art, including, but not limited to alprazolam, bromazepam, clonazepam, diazepam, estazolam, flunitrazepam, lorazepam, midazolam, nitrazepam, oxazepam, triazolam, temazepam, and chlordiazepoxide. The compounds disclosed herein can also be administered in combination with other classes of compounds, including, but not limited to, endothelin converting enzyme (ECE) inhibitors, such as phosphoramidon; thromboxane receptor antagonists, such as ifetroban; potassium channel openers; thrombin inhibitors, such as hirudin; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-adrenergic agents; beta-adrenergic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone antagonists, and octreotide acetate; microtubule-disruptor agents, such as ecteinascidins; microtubule-stabilizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic. In some embodiments, the kit or article of manufacture includes a container (such as a bottle) with a desired amount of at least one compound (or pharmaceutical composition of a compound) as disclosed herein.

For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use.

Such a kit or article of manufacture can further include instructions for using said compound (or pharmaceutical composition of a compound) disclosed herein. In some embodiments, a set of instructions is included. In other embodiments, the instructions are attached to the container, or are included in a package (such as a box or a plastic or foil bag) holding the container.

In another embodiment, the kit or article of manufacture is a tamper resistant kit or article of manufacture.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

This invention will be better understood by reference to the Experimental section which follows, but those skilled in the art will readily appreciate that the specific experiments detailed are only illustrative of the invention as described more fully in the claims which follow thereafter.

### EXAMPLE 1

### Synthesis of (2S)-2-(2-chlorophenyl)-6,6-dideuterio-2-[deuterio(methyl)amino]cyclohexanone, deuterium chloride(D2-(S)-ketamine DCl

A. Route 1

To a 500 mL single neck flask, equipped with a stirrer bar, thermocouple, and nitrogen line, was charged 4.9695 g (18.1 mmol) of (S)-(+)-ketamine HCl salt, 100 mL of ethyl acetate and 100 mL of NaHCO₃ saturated aqueous solution. The mixture was stirred for 10 minutes at room temperature before transferring into a separation funnel. After partition, the bottom aqueous layer was back-extracted with additional 100 mL of ethyl acetate. The organic layers were combined and dried over anhydrous sodium sulfate. This was filtered, and the filtrate was concentrated to dryness, affording 4.2023 g (17.7 mmol) of (S)-ketamine as a white solid, representing a 97.8% yield in 99.4% purity.

A 25 mL sealed tube was charged with 0.25 g (1.05 mmol) of esketamine free amine (2), 2.5 mL of CD₃OD, 9 mL of D₂O and 1 mL of NaOD in D₂O (40 wt %). After capping the tube, the mixture was heated to 40° C. for 14-24 hours. The resultant mixture was extracted with 3×10 mL of ethyl acetate. The combined extractions were concentrated to dryness. The residue was re-subjected to the same reaction for additional two times, allowing the D-H exchange to be over 98% determined by ¹H NMR analysis. The residue was dissolved in 40 mL of anhydrous ethyl ether, filtered on a Buchner filter with a fine frit into a 250 mL three neck flask. The filter cake was washed with 10 mL of ethyl ether, the wash was combined with the filtrate. DCl gas was blown over the surface of the solution with stirring. The product precipitated, and the slurry was stirred at room temperature for 1 hour before filtering on a Buchner funnel. The filter cake was washed with 2×10 mL of ethyl ether, and dried under vacuum overnight at room temperature, affording 0.2017 g of d2-(S)-ketamine DCl salt (formula Ib DCl salt), representing a 69.6% yield in 100 A% chemical purity and >99% ee. LC-MS analysis indicated the deuterium incorporation was 96.0% D2, 2.7% DL ¹H NMR (CDCl₃, 400 MHz): δ=1.57-1.64 (m, 1H), 1.84-1.91 (m, 2H), 2.02-2.04 (m, 1H), 2.47-2.57 (m, 1H), 2.58-2.60 (m, 3H), 3.57-3.58 (m, 1H), 7.46-7.48 (m, 2H), 7.50-7.57 (m, 1H), 8.04 (d, J=8.0 Hz, 1H), 9.53 (brm, 1H), 10.71 (brm, 1H); HRMS-ESI (m/z): [M+H]⁺ Calcd for C₁₃H₁₄D2ClNO: 240.1046; found 240.1126.

Changes in the metabolic properties of the compounds of Example 1 and its analogs as compared to its non-isotopically enriched analogs can be shown using the following assays. Other compounds listed above, which have not yet been made and/or tested, are predicted to have changed metabolic properties as shown by one or more of these assays as well.

B. Route 2

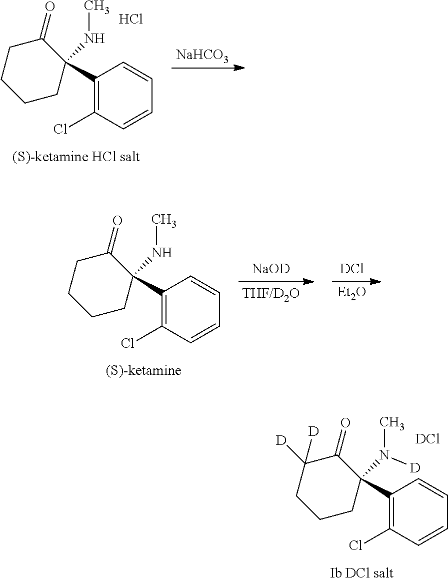

A 1000 ml, round bottom flask, equipped with a stir bar and nitrogen, was charged with 23.82 g (0.0915 mol, 1.0 eq) of (S)-ketamine HCl. The flask was charged with 450 mL (20V) of ethyl acetate, and 450 mL (20V) of a saturated sodium bicarbonate solution. The mixture was stirred for 15 minutes. The mixture was poured into a separatory funnel, and the layers were separated. The aqueous layer was washed with 450 mL of ethyl acetate. The organic layers were combined and dried over Na₂SO₄. The solution was filtered, and concentrated to dryness. Obtained was 20.05 g of (S)-ketamine, representing a 98% yield, and 100 A% purity.

A 500 mL round bottom flask, equipped with a stir bar, and nitrogen, was charged with 5.15 g (0.0216 mol, 1.0 eq) of (S)-ketamine and 25 ml (5V) of anhydrous THF, and stirred until all the solids went into solution. Then was added 100 mL (20 V) of deuterium oxide, and 20 mL (4V) of sodium deuterate. The reaction was heated at 65° C. for 24 hours. The reaction was complete with 24 hours, and checked by taking an aliquot from the reaction, and running an NMR. The reaction was cooled to room temperature, the mixture was poured into a separatory funnel and extracted with ethyl acetate (3×100 mL). Concentrated the organic layer to dryness. Obtained 4.64 g of d2-(S)-ketamine free base with 89.2% yield.

A 250 mL, three neck round bottom flask equipped with a stir bar and nitrogen, was charged with 2.31 g (9.64 mmol, 1.0 eq) of D2-(S)-ketamine free base and 70 mL (30 V) of diethyl ether was added. The reaction was placed into an ice bath, and cooled to 0° C. DCl gas was added. A white precipitate started forming in the flask. The completion of the reaction was monitored by testing the pH of the reaction, an acidic reaction deemed the reaction complete. After the addition of DCl gas was complete, the ice bath was removed and the reaction was allowed to warm to room temperature, and stirred for 1 hour. After 1 hour, the reaction was filtered through a sintered funnel, and the solids were washed with 20 mL (10 V) of diethyl ether. The white solid was dried under vacuum with a canopy of nitrogen. Obtained was 2.44 g of alpha d2-(S)-ketamine DCl salt (formula Ib DCl salt) in a 90% yield, 100 A% purity, and 1.6% D1, 98.4% D2, 0.0% D3.

### EXAMPLE 2

### In Vitro Liver Microsomal Stability Assay

Liver microsomal stability was measured using 0.5 mg per mL of liver microsomal protein with a NADPH-generating system (1 mM NADPH, 5 mM glucose 6-phosphate and 1 unit per mL glucose 6-phosphate dehydrogenase) in potassium phosphate buffer (50 mM, pH 7.4) containing MgCl₂ (3 mM) and EDTA (1 mM, pH 7.4). Test articles were added for a final assay concentration of 1 μM and incubated at about 37° C. Reactions were started by addition of the cofactor, and were stopped at four designated time points (0, 15, 30 and 60 min) by the addition of an equal volume of stop reagent (e.g., acetonitrile containing an internal standard, 0.2 mL). Samples were then centrifuged at 920×g centrifugal force for 10 min at 10° C. to precipitate the proteins. Supernatants were analysed by LC/MS/MS. It has thus been found that the compounds as disclosed herein according to the present disclosure that have been tested in this assay showed an increase of 10% or more in the degradation half-life, as compared to the non-isotopically enriched drug. The degradation half-lives of Example 1 were increased by 18%, as compared to non-isotopically enriched ketamine.

### EXAMPLE 3

### Stability of alpha-d2-(R/S)-ketamine (Example 1 Racemate) in Phosphate-Buffered Saline at Various pH Levels

The stability of alpha-deuterium-substituted (d2) (R/S)-ketamine (Example 1 as a racemate) in phosphate-buffered saline at pH 2.0, 7.4 and 8.4 at 37±1° C. was evaluated. Incubations of (d2) (R/S)-ketamine (e.g., 1 μM) with PBS (pH 2.0, 7.4 and 8.4) were carried out using a Tecan Liquid Handling System (Tecan), or equivalent, at 37±1° C. in 0.2-mL incubation mixtures (final volume) containing PBS, at the final concentrations indicated in a 96-well plate format. The test article was added to the incubation mixtures in water. Reactions were started by addition of the test article, and stopped at four designated time points (e.g., 0, 30, 60 and 120 min) by the addition of an equal volume of stop reagent (e.g., acetonitrile, 0.2 mL containing an internal standard). Incubations were carried out in triplicate with an exception for zero-time samples (which were incubated in quadruplicate). The samples were centrifuged (e.g., 920×g for 10 min at 10° C.) and the supernatant fractions analyzed by LC-MS/MS. Non-deuterated (d0) (R/S)-ketamine was used as an internal standard. The amount of unchanged test article and formation of non-deuterated (d0) (R/S)-ketamine was monitored based on peak area ratio of the analyte/internal standard.

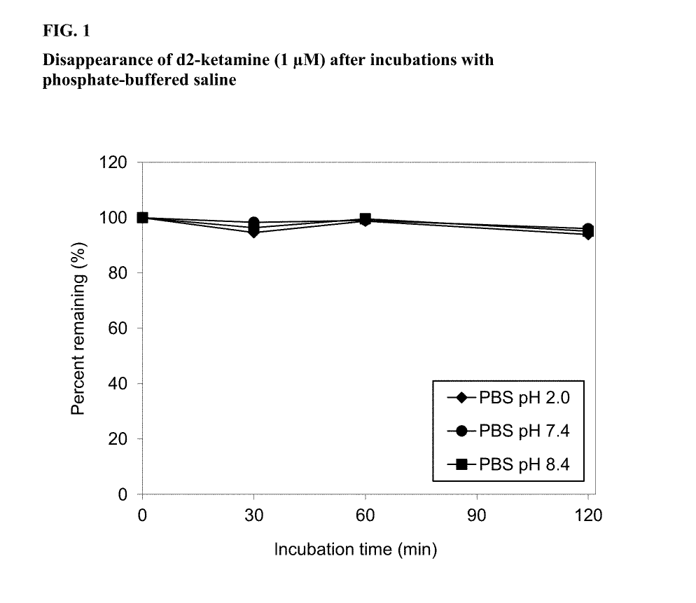

Data are shown below in Table 1 and 2. The data for Table 1 are presented graphically in FIG. 1.

TABLE 1
Incubations of d2-ketamine with phosphate-buffered saline

| | Mean percent | Mean percent | Mean percent | Mean percent |
|---|---|---|---|---|
| PBS | remaining | remaining | remaining | remaining |
| pH | (0 min) | (30 min) | (60 min) | (120 min) |
| 2.0 | 100% | 94.6% | 98.8% | 93.9% |
| 7.4 | 100% | 98.3% | 99.0% | 96.0% |
| 8.4 | 100% | 96.4% | 99.6% | 95.1% |

TABLE 2
Incubations of d2-ketamine with phosphate-buffered saline
(analyte D0-ketamine)

| | Mean area | Mean area | Mean area | Mean area |
|---|---|---|---|---|
| | ratio | ratio | ratio | ratio |
| PBS pH | (0 min) | (30 min) | (60 min) | (120 min) |
| 2.0 | NA | ND | ND | ND |
| 7.4 | NA | ND | ND | ND |
| 8.4 | NA | 0.00367 | ND | ND |

Mean area ratio values were blank corrected to subtract the detection in zero-minute samples.
NA Not applicable
ND Not detected (value was zero or negative)

### EXAMPLE 4

### In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound as disclosed herein, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### EXAMPLE 5

### Monoamine Oxidase A Inhibition and Oxidative Turnover

The procedure is carried out as described in Weyler, Journal of Biological Chemistry 1985, 260, 13199-13207. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaP, buffer, pH 7.2, containing 0.2% Triton X-100 (monoamine oxidase assay buffer), plus 1 mM kynuramine, and the desired amount of enzyme in 1 mL total volume.

### EXAMPLE 6

### Monoamine Oxidase B Inhibition and Oxidative Turnover

The procedure is carried out as described in Uebelhack, Pharmacopsychiatry 1998, 31, 187-192.

### EXAMPLE 7

### Inhibition of [³H]TCP Binding to the Rat NMDA Receptor

The procedure is carried out as described in Goldman et al, FEBS Letters 1985, 190(2), 333-336.

### EXAMPLE 8

### Rat Model for Hypoxia-Induced Neurodegeneration and NMDA-Antagonist Neuroprotection

The procedure is carried out as described in Reeker et al, Canadian Journal of Anaesthesia 2000, 37(6), 572-578.

### EXAMPLE 9

### Determination of the In Vitro Metabolism of S-Ketamine and alpha d2 S-ketamine in Microsomes

A. Incubation Conditions

S-Ketamine and alpha d2 S-Ketamine (10 μM) were incubated with liver microsomes (2 mg/mL in 0.1 M potassium phosphate buffer containing 1 mM EDTA, assay buffer) for 0 and 15 minutes (rat) or 0 and 30 minutes (human) at 37° C. Incubations were initiated by the addition of nicotinamine adenine dinucleotide phosphate (NADPH, 1 mM) and terminated by the addition of methanol. Samples were vortex mixed, stored on ice, and the samples were centrifuged at 1400×g for 5 minutes. Supernatants were removed from the microsome pellets and stored in new tubes at approximately −20° C. until analysis. Metabolic controls were conducted by incubating S-ketamine (10 μM) in microsomes (2 mg/mL) in the absence of NADPH at 0 and 15 or 30 minutes, respectively, to determine the stability of the test article under the incubation conditions.

B. Characterization of Metabolites

Metabolites generated in microsome incubation samples were characterized by LC-MS/MS using a LTQ Orbitrap XL with electrospray ionization in positive ion mode. Semi-quantitation of the metabolites present in the samples was based on LC MS peak areas of the putative metabolites. The structural elucidation of metabolites of S-ketamine and the d2 S-ketamine was accomplished through the use of standards (6-OH-norketamine, norketamine and dehydronorketamine), references to reported metabolites in the scientific literature (Turfus et al., 2009; Bijlsma et al., 2011), high resolution accurate mass spectrometry, interpretation of product ion spectra and comparison of chromatographic retention times among metabolites.

TABLE 3
% Peak Area after incubation of S-ketamine and d2 S-ketamine
in rat and human liver microsomes

| | Rat liver | | Human liver | |
|---|---|---|---|---|
| | microsomes (15 min) | | microsomes (30 min) | |
| | S- | d2 S- | S- | d2 S- |
| Component | ketamine | ketamine | ketamine | ketamine |
| S-ketamine | 4.6 | 2.6 | 31.2 | 33.6 |
| 6-OH-ketamine | 0.8 | ND | 3.9 | 1.0 |
| OH-ketamine | ND | 0.1 | ND | ND |
| OH-ketamine1 | 6.3 | 3.7 | 0.2 | 0.4 |
| OH-ketamine2 | ND | ND | ND | 0.1 |
| norketamine | 35.1 | 75.9 | 59.8 | 60.8 |
| 6-OH-norketamine | 40.7 | 7.1 | 1.6 | 0.7 |
| OH-norketamine1 | 0.9 | ND | ND | ND |
| OH-norketamine2 | 10.5 | 8.3 | 2.0 | 1.5 |
| OH-norketamine3 | ND | 1.7 | ND | 0.4 |
| dehydro-norketamine1 | 1.3 | 0.5 | 0.1 | ND |
| dehydro-norketamine2 | ND | 0.1 | ND | ND |
| phenol-ketamine1 | ND | ND | 0.2 | 0.1 |
| phenol-ketamine2 | ND | ND | 0.9 | 1.1 |
| phenol-norketamine | ND | ND | 0.1 | 0.1 |

ND = Not detected or below the limit of quantitation (0.1% of the total chromatographic peak area)
¹deuterated analog for incubations with d2S-ketamine

C. Results

In rat liver microsomal incubation, S-ketamine and its d2 analog were extensively metabolized, with approximately 5 and 3% of the parent compound remaining, respectively, after 15 minutes. In human liver microsomal incubation, catalytic rates were lower compared to rat liver microsomal incubations with approximately 31 and 34% of the parent compound remaining, respectively, after 30 minutes. The major metabolites formed from S-ketamine by rat liver microsomes were norketamine and 6-OH-norektamine with 35.1 and 40.7%, respectively. The deuteration at the 6-position in d2 S-ketamine resulted in a marked decrease in formation of the 6-OH-norketamine analog indicating that the deuterium abstraction is the rate-limiting step for the 6-hydroxylation reaction. Alternate hydroxylation sites were not favored and therefore norketamine was the dominant metabolite in rat liver microsomal incubations of d2 S-ketamine with 75.9%.

In human liver microsomal incubations, norketamine was the dominant metabolite formed from both S-ketamine and d2 S-ketamine. Apparently, 6-OH-hydroxylation is a slow process in liver microsomal incubations and only small amounts were observed, although it is a major circulating metabolite in human in vivo. Because only small amounts of 6-OH-ketamine and 6-OH-norketamine were formed from S-ketamine under the test conditions, the impact of the deuteration at the 6-position was only marginally apparent in human liver microsomes.

### EXAMPLE 10

### Tail Suspension Test (Mice)

Study Objective: The Tail Suspension Test (TST) is a means of evaluating potential antidepressants. The immobility displayed by rodents when subjected to an unavoidable and inescapable stress has been hypothesized to reflect behavioral despair which in turn may reflect depressive disorders in humans. Clinically effective antidepressants reduce the immobility that mice display after active and unsuccessful attempts to escape when suspended by the tail.

In this study, the efficacy of D-2, D-6, and D-8 esketamine in the TST mouse model of depression were tested. The results were compared to the extent of the effect of esketamine.

Study Design and Procedure: Groups of 10 animals were treated with the test compounds or the vehicle control 30 minutes before testing, by intraperitoneal (ip) injection. For the test, the mice were suspended on the edge of a shelf (closet) ˜75 cm above the table top by adhesive tape placed approximately 1 cm from the tip of the tail. The duration of immobility is recorded for a period of 6 min. Mice were considered immobile when they hanged passively and completely motionless.

Mice were weighed and randomly assigned to groups. The formulations and dose levels were prepared blinded to administration and test recording. The blinded coding was kept concealed by the formulator and the team leader. The data was exposed for analysis and reporting.

Test Animals: Animals/Group Size:

90 Mice in the study (C57/Bl/6). 10 mice/group (male; 21-24 g) on arrival. Mice were kept at reversed light cycle and were tested in the morning time 08:00-13:00.

Test Compounds, Dose Levels, and Route of Administration:

Test Articles:
S-ketamine HCl (esketmine HCl) (86.5% s-ketamine (esketamine) base).
D-2 (S)(+)ketamine DCl Salt, (87.0% s-ketamine base).
D-6 (S)(+)ketamine DCl Salt, (87.0% s-ketamine base).
D-8 (S)(+)ketamine DCl Salt, (87.0% s-ketamine base).
Saline

Compound Preparation and Dosing: Test compounds and grouping was code-labeled. S-ketamine salt and deuterated (S)-ketamine DCl (D-2, D-6, and D-8) were dissolved in 10 mL saline. The lower doses were prepared by serial dilutions. The dose volume of all treatments was 10 mL/kg.

Treatment Group Design:
1. Control—Saline 10 ml/kg ip 30 min before test (n=10)
2. Esketamine 15 mg/kg ip 30 min before test (n=10)
3. Esketamine 30 mg/kg ip 30 min before test (n=10)
4. D2 15 mg/kg ip 30 min before test (n=10)
5. D2 30 mg/kg ip 30 min before test (n=10)
6. D6 15 mg/kg ip 30 min before test (n=10)
7. D6 30 mg/kg ip 30 min before test (n=10)
8. D8 15 mg/kg ip 30 min before test (n=10)
9. D8 30 mg/kg ip 30 min before test (n=10)

Test compounds were administered half an hour before suspending the animals by the tail.

Evaluation: The duration of immobility was the measured parameter. This was calculated from the cumulated time during the 6 minute period the animals were suspended. Immobility time of each set of animals was recorded manually with stopwatches by three individuals. Animals were also recorded with the NOLDUS video tracking system.

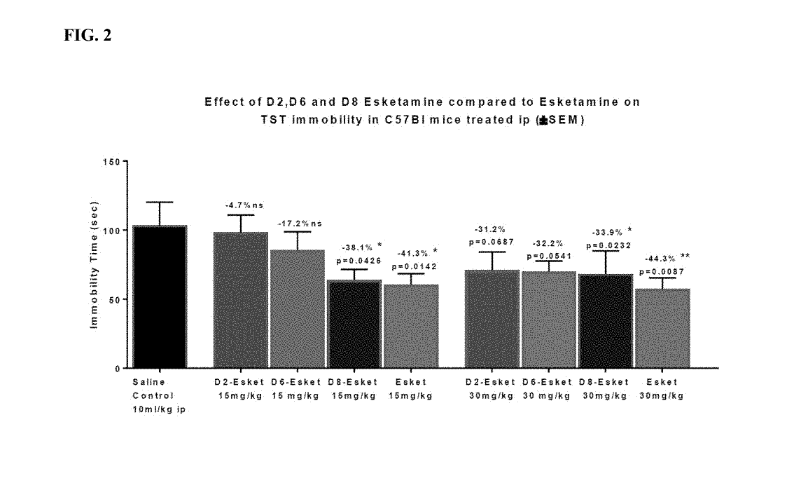

Statistical evaluation was done with ordinary One-Way ANOVA, followed by Uncorrected Fisher's LSD test (Saline and WFI as vehicle control groups). The graphing (FIG. 2) and statistics was evaluated with GraphPad Prism 6 statistical program.

Results: Inbred C57Bl/6 strain mice were treated with Esketamine (S-isomer of ketamine), D-2, D-6, and D-8 (S)-ketamine (esketamine) DCl at dose levels of 15 and 30 mg/kg ip. Saline was used as vehicle controls. The injected groups were exposed to the tail suspension test (TST) 30 minutes after treatment. The D-8 (S)(+)ketamine and the Esketamine exhibited statistical significant efficacy in reducing immobility time when treated ip at both doses of 15 and 30 mg/kg. Esketamine at dose levels of 15 and 30 mg/kg ip reduced the immobility time by −41.3% (p=0.0142) and -−44.3% (p=0.0087), respectively, statistically significant when compared to the saline control group. D-8 Esketamine at 15 and 30 mg/kg ip also reduced immobility time by −38.1% (p=0.0426) and −33.9% (p=0.0232), respectively. The D-2 and D-6 esketamine induced a similar trend as Esketamine at both doses tested but did not reach p<0.05 (See FIG. 2).

### EXAMPLE 11

### Forced Swim Test (Mice)

Study Objective. To evaluate anti-depressant-like activity by evaluating immobility time of mouse despaired to escape from a cylinder filled with water, a situation considered to mirror the depressed behavior in humans.

Study Design. In two sessions separated by 24 hours, 90 mice were forced to swim in a cylinder from which they could not escape. The first 15-min session was conducted prior to drug administration and without behavioral recording. This prior habituation session ensures a stable and high duration of immobility during the 5-min test session performed on the next day. 30 min before the test, mice were treated i.p. with saline (control group) or one of 4 tested compounds at doses of 15 or 30 mg/kg each. During the test, the immobility time was recorded manually and by videorecorder.

Test Articles.
Water for injection
S-ketamine HCl (86.5% s-ketamine base).
D-2 S-ketamine
D-6 S-ketamine
D-8 S-ketamine

Compound Preparation and Dosing.

The volume of all treatments was 10 mL/kg.

| Treatment | Amount (cc) | Concentration (salt) |
|---|---|---|
| Saline | 4 | 0.9% |
| Esketamine | 4 | 3.5 mg/mL |
| Esketamine | 4 | 1.75 mg/mL |
| D2-esketamine | 4 | 3.5 mg/mL |
| D2-esketamine | 4 | 1.75 mg/mL |
| D6-esketamine | 4 | 3.5 mg/mL |
| D6-esketamine | 4 | 1.75 mg/mL |
| D8-esketamine | 4 | 3.5 mg/mL |
| D8-esketamine | 4 | 1.75 mg/mL |

Test Animals. 90 male C57Bl mice 6 weeks old at receiving, obtained from Envigo animal breeding center. Animals housing and care conditions were maintained according to SOPs 33.22.01 and 33.22.02. Rats were kept at reversed light cycle and were tested in the morning time 08:00-13:00.

Experimental Design. Each mouse was placed for 15 min in the cylinder filled with the tap water (23° C.) with the depth of 10 cm. Immediately after the session, the mouse was allowed to keep warm and to dry up in the cage under the red light. On the next day, 30 min following the treatment, each mouse was placed in the cylinder under the same conditions for 5 min and immobility time (the animal “freezes” in the water) was recorded.

Groups and Treatments.

The study was divided into two experimental parts in order to make possible testing 90 animals in the morning hours. Each group was represented equally on both days. Test solutions were coded and the researcher was blinded to the content of vials.

| Group | n | Dose | Route | Test following treatment |
|---|---|---|---|---|
| Saline | 10 | 4 mL/kg | i.p. | 30 min. |
| Esketamine | 10 | 15 mg/kg | i.p. | 30 min. |
| Esketamine | 10 | 30 mg/kg | i.p. | 30 min. |
| D2-esketamine | 10 | 15 mg/kg | i.p. | 30 min. |
| D2-esketamine | 10 | 30 mg/kg | i.p. | 30 min. |
| D6-esketamine | 10 | 15 mg/kg | i.p. | 30 min. |
| D6-esketamine | 10 | 30 mg/kg | i.p. | 30 min. |
| D8-esketamine | 10 | 15 mg/kg | i.p. | 30 min. |
| D8-esketamine | 10 | 30 mg/kg | i.p. | 30 min. |

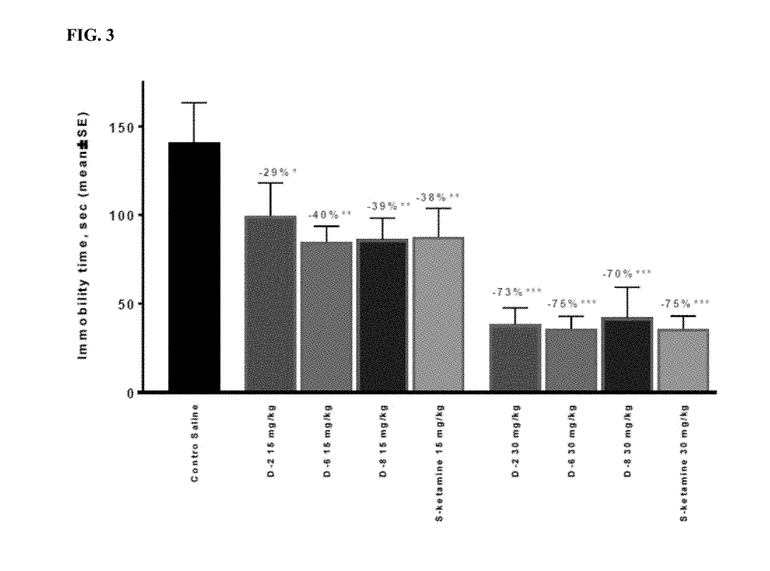

Results. The comparison in immobility time between the groups was done using One-Way Anova followed by Tukey post-hoc test. S-Ketamine, as well as the deuterated analogs, were markedly effective in reducing immobility time comparing to controls, at both doses tested. The effect following 15 mg/kg dose treatment ranged between 29-40% and after 30 mg/kg—between 70-75%. Each deuterated S-ketamine derivative (D2, D6, and D8) showed a similar potency as Esketamine in the rat FST model. See, FIG. 3.

### EXAMPLE 12

### Forced Swim Test (Rats)

Study Objective. To evaluate anti-depressant-like activity by the immobility time of rat despaired to escape from a cylinder filled with water, a situation considered to mirror the depressed behavior in humans.

Study Design. In two sessions separated by 24 hours, 94 rats were forced to swim in a cylinder from which they could not escape. The first 15-min session was conducted prior to drug administration and without behavioral recording. This prior habituation session ensures a stable and high duration of immobility during the 5-min test session performed on the next day. 75-90 min before the test, rats were treated orally with WFI (control group) or one of 3 tested compounds at doses of 60 or 120 mg/kg each. During the test, the immobility time was recorded manually and by video recorder.

Test Articles
Water for Injection
Ketamine hydrochloride (solution for injection 100 mg/ml).
S-ketamine HCl (86.5% s-ketamine base).
D-2 S-ketamine
D-6 S-ketamine

Compound preparation and dosing. On each test day, 280.9 mg of S-ketamine salt (deuterated or non-deuterated) equivalent to 243 mg active compound was dissolved in 8.1 ml WFI to yield 30 mg base/1 ml solution (120 mg/4 ml/kg). For 60 mg/kg dose solution, this solution was divided 1:2 (2.7 ml+2.7 ml WFI). The volume of all treatments was 4 ml/kg.

Test animals. 94 male SD male rats 6 weeks old at receiving, obtained from Harlan animal breeding center. Animals housing and care conditions were maintained according to SOPs 33.22.01 and 33.22.02. Rats were kept at reversed light cycle and were tested in the morning time 08:00-13:00.

Experimental Design. Each rat was placed for 15 min in the cylinder filled with the tap water (23° C.) with the depth of 30 cm. Immediately after the session, the rat was allowed to keep warm and to dry up in the cage under the red light. On the next day, 30 min (ketamine I.P.) or 90 min (all the oral treated groups) following the treatment, each rat was placed in the cylinder under the same conditions for 5 min and immobility time (the animal “freezes” in the water) was recorded. After the calculations of the first day it was decided to reduce the time to test to 75 min on the second testing day. The study was divided into two experimental parts with 47 rats each in order to make possible testing 94 animals in the morning hours. Each group was represented equally on both days.

| | | | | | Test following |
|---|---|---|---|---|---|
| Group | n | Dose | | Route | treatment |
| Water for Inj. | 12 | 4 | mL/kg | p.o. | 90 min or 75 min |
| Esketamine | 11 | 60 | mg/kg | p.o. | 90 min or 75 min |
| Esketamine | 11 | 120 | mg/kg | p.o. | 90 min or 75 min |
| D2-esketamine | 12 | 60 | mg/kg | p.o. | 90 min or 75 min |
| D2-esketamine | 12 | 120 | mg/kg | p.o. | 90 min or 75 min |
| D6-esketamine | 11 | 60 | mg/kg | p.o. | 90 min or 75 min |
| D6-esketamine | 11 | 120 | mg/kg | p.o. | 90 min or 75 min |
| ketamine | 8 | 30 | mg/kg | i.p. | 30 min |

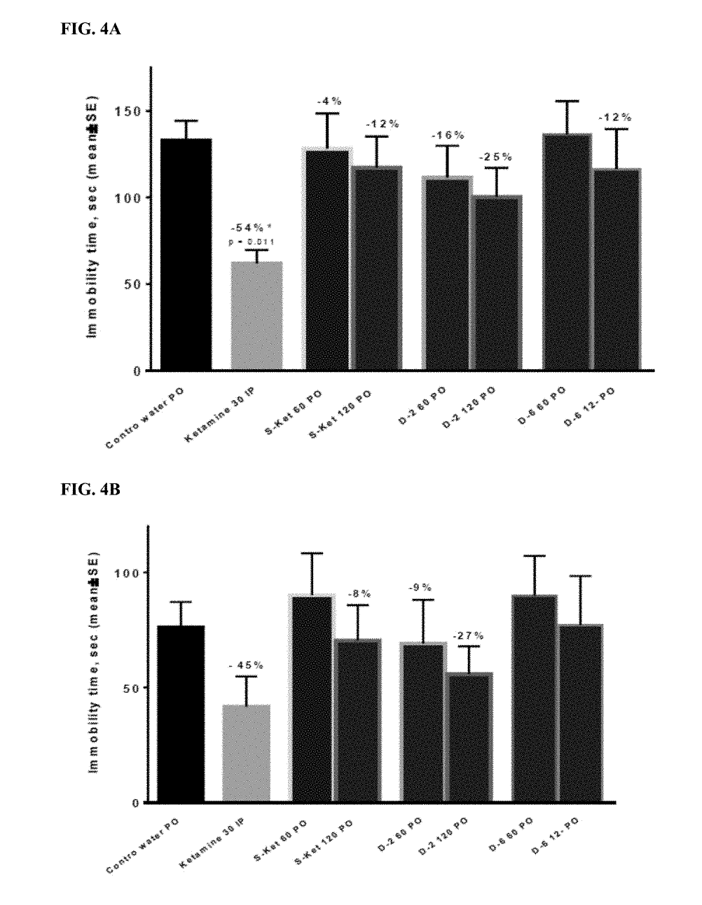

Results. Results of both testing days were combined for calculations. The comparison in immobility time between the groups was done using One-Way Anova followed by LSD post-hoc test. D-2 S-ketamine showed dose-response trend. None of the 3 compounds given by oral route reduced significantly the immobility time as compared to controls. The positive control ketamine 30 mg/kg i.p. was very effective (54% reduction by manual counting, p=0.0105). See FIGS. 4A and 4B. S-ketamine and its deuterated analogs given orally did not show antidepressant-like effect in rat FST model when tested within 75-90 min after the treatment. D-2 S-ketamine produced slight reduction in immobility time (˜25% at 120 mg/kg dose).

### EXAMPLE 13

### Tail Suspension Test (Mice)

Study Summary. Esketamine and D-2 esketamine were intraperitoneally (ip) and orally (po) administered to inbred C57Bl/6 strain mice. The anti-depressive activity in the TST showed a dose response in both esketamine and its deuterated form D-2. D-2 esketamine exhibited an overall higher reduction in percent immobility time than the Esketamine.

Esketamine and D-2 esketamine (120 mg/kg p.o.) decreased immobility time to its peak effect and was most highly significant when compared to vehicle WFI-treated animals. Prior to testing at this dose level animals were accompanied with adverse clinical signs characterized to the ketamine anasthetic dose effect.

Ketamine at 30 mg/kg ip served as positive reference control administered 30 minutes prior to testing exhibited an anti-depressant like effect in the TST but was less effective, percent-wise, than the deuterated form.

Study Objective. This study is to assess the potential efficacy of the isomer esketamine and its deuterated D-2 analog in a mouse model of depression and to compare the extent of the effect to that of ketamine racemate.

The Tail Suspension Test (TST) is a means of evaluating potential antidepressants. The immobility displayed by rodents when subjected to an unavoidable and inescapable stress has been hypothesized to reflect behavioral despair which in turn may reflect depressive disorders in humans. Clinically effective antidepressants reduce the immobility that mice display after active and unsuccessful attempts to escape when suspended by the tail.

Commercially supplied ketamine, a mixture of the R+ and S− isomers, was tested and compared it to the S− isomer and deuterated D-2 form. Three dose levels of the esketamine and D-2 esketamine each were administered by ip and oral gavage to inbred C57Bl/6 strain mice. Efficacy was evaluated in comparison to Saline 0.9% and water for injection (WFI) vehicle control group. The potency of the esketamine and D-2 esketamine were compared to the ip injection of the ketamine racemate, which served positive reference.

Study Design and Procedure. Groups of 8, 10, or 12 animals were treated with the test compounds or the vehicle and positive control by ip injection or oral (P.os) administration 30 and 45 minutes respectively prior to testing. For the test the mice were suspended on the edge of a shelf (closet) ˜75 cm above the table top by adhesive tape place approximately 1 cm from the tip of the tail. The duration of immobility was recorded for a period of 6 min. Mice were considered immobile when they hang passively and completely motionless.

Test Animals.
100 Mice in the study (C57Bl/6)
10 or 15 C57Bl/6/group (male; 21-24 g) on arrival
Mice were kept at reversed light cycle, 12 h/12 h dark/light cycle (lights on 19:00) and were tested in the morning time 08:00-13:00.

Test articles and Compound Preparations.
Ketamine hydrochloride (solution for injection 100 mg/ml).
S-ketamine HCl (86.5% s-ketamine base).
Deuterated (S)(+)ketamine DCl (D-2) (86.5% s-ketamine base).
WFI (Water for injection)
Saline

Ketamine salt solution was freshly prepared (0.3 ml of original solution was added to 9.7 ml saline) and injected i.p. 30 min before the test. 138.7 mg of S-ketamine salt and Deuterated (S)(+)ketamine DCl (D-2), was dissolved in 10 ml WFI each. The lower doses were prepared by serial dilutions. The dose volume of all treatments were administered at 10 ml/kg. Dose Levels and Route of Administration

Test Compounds were administered half and ¾ of an hour as designated by the test group before suspending the animals by the tail.
1. Control—WFI 10 ml/kg p.o. 45 min before test (n=10)
2. Control—Saline 10 ml/kg ip 30 min before test (n=10)
3. Ketamine 30mg/kg ip 30 min before test (n=8)
4. Esketamine 15 mg/kg ip 30 min before test (n=12)
5. Esketamine 30 mg/kg ip 30 min before test (n=12)
6. D2 15 mg/kg ip 30 min before test (n=12)
7. D2 30 mg/kg ip 30 min before test (n=12)
8. Esketamine 120 mg/kg p.o. 45 min before test (n=12)
9. D2 120 mg/kg p.o. 45 min before test (n=12)

Evaluation. The duration of immobility was the measured parameter. This was calculated from the cumulated time during the 6 minute period the animals were suspended. Immobility time of each set of animals was recorded manually with stopwatches by three individuals. Animals were also recorded with the NOLDUS video tracking system. Statistical evaluation was done with ordinary One-Way ANOVA, followed by Uncorrected Fisher's LSD test (Saline and WFI as vehicle control groups). The graphing and statistics were evaluated with GraphPad Prism 6 statistical program.

Results. Inbred C57Bl/6 strain mice were treated with Esketamine (S-isomer) and D-2 (S)(+)ketamine DCl at dose levels of 15, 30 mg/kg ip and 120 mg/kg oral gavage. Saline and WFI were used as vehicle controls, while Ketamine (R+ and S− isomers) served as a positive control group. The intraperitoneally injected groups were exposed to the tail suspension test (TST) 30 minutes after treatment, while the orally gavaged animals were tested after 45 minutes. Both compounds exhibited efficacy in reducing immobility time when treated ip.

The oral gavage treatments of Esketamine and D-2 esketamine 120 mg/kg were profoundly active but were also accompanied with adverse clinical symptoms of vocalization, ataxia and drowsiness. During the 6 minute test session when mice were suspended by the tail, the movements in the oral 120 mg/kg treatments were more of in tremor form involving trembling and quivering of the limbs or entire body. This was not like the bending of the body upwards to counteract the hanging down when suspended of the lowered dosed animals.

One of the animals was found dead in its cage 20 minutes after the oral gavage from the esketamine 120 mg/kg treatment group.

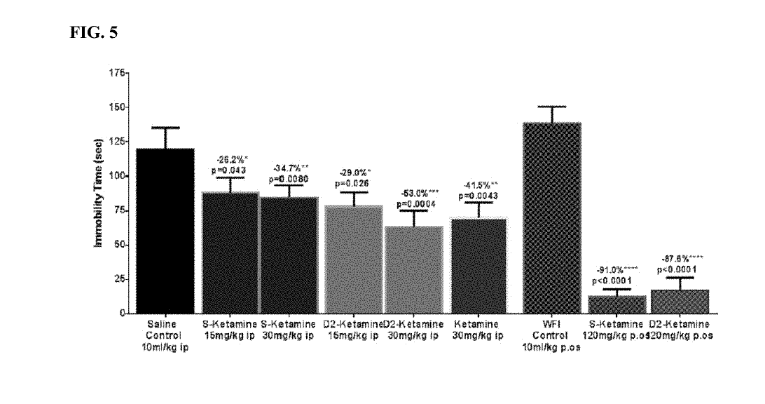

The Ketamine racemate positive control at 30 mg/kg lowered the immobility time by −41.5% from the saline control group. The Esketamine at dose levels of 15 and 30 mg/kg ip slightly reduced the immobility time by −26.2% and −34.7%, respectively, statistically significant when compared to its respective saline control group. In comparison, D-2 Esketamine at 15 and 30 mg/kg ip also reduced immobility time by −29.0% and −53.0% respectively, but more pronounced than the esketamine. The esketamine and D-2 esketamine at the oral administration of 120 mg/kg decreased immobility time by −91.0% and −87.6% respectively, highly statistically significant. See FIG. 5.

Conclusion. S-ketamine salt and Deuterated (S)(+)ketamine DCl was intraperitoneally (ip) and orally administered to inbred C57Bl/6 strain mice. The anti-depressive activity in the TST showed a dose response in both esketamine and its deuterated form D-2. D-2 esketamine exhibited a higher reduction in percent immobility time than the Esketamine. Esketamine and Deuterated (S)(+)ketamine both at 120 mg/kg oral gavage decreased immobility more dramatically when compared to vehicle-only treated animals, although untoward clinical signs were expressed. It may be concluded these compound formulations show anti-depressant efficacy.

### EXAMPLE 14

### Forced Swim Test (Rats)

Study Objective. To evaluate the anti-depressant-like activity by immobility time of rat despaired to escape from a cylinder filled with water, a situation considered to mirror the depressed behavior in humans.

Study Design. In two sessions separated by 24 hours, 90 rats were forced to swim in a cylinder from which they could not escape. The first 15-min session was conducted prior to drug administration and without behavioral recording. This prior habituation session ensures a stable and high duration of immobility during the 5-min test session performed on the next day. 30 min before the test, rats were treated orally with WFI (control group) or one of 3 tested compounds at doses of 15 or 30 mg/kg each. During the test, the immobility time was recorded manually and by videorecorder.

Test Articles.
WFI
S-ketamine HCl (86.5% s-ketamine base).
D-2 S-ketamine
D-6 S-ketamine
D-8 S-ketamine

Compound Preparation and Dosing.

The compounds were prepared by a chemist and coded, so the researcher was blinded to the treatment. The volume of all treatments was 4 mL/kg.

| Treatment | Amount (cc) | Concentration (salt) |
|---|---|---|
| Saline | 12 | 0.9% |
| Esketamine | 12 | 8.675 mg/mL |
| Esketamine | 12 | 4.34 mg/mL |
| D2-esketamine | 12 | 8.675 mg/mL |
| D2-esketamine | 12 | 4.34 mg/mL |
| D6-esketamine | 12 | 8.675 mg/mL |
| D6-esketamine | 12 | 4.34 mg/mL |
| D8-esketamine | 12 | 8.675 mg/mL |
| D8-esketamine | 12 | 4.34 mg/mL |

Test Animals. 90 male SD male rats 6 weeks old at receiving, obtained from Harlan animal breeding center. Animals housing and care conditions were maintained according to SOPs 33.22.01 and 33.22.02. Rats were kept at reversed light cycle and tested in the morning time 08:00-13:00.

Experimental Design. Each rat was placed for 15 min in the cylinder filled with the tap water (23° C.) with the depth of 30 cm. Immediately after the session, the rat was allowed to keep warm and to dry up in the cage under the red light. On the next day, 30 min following the treatment, each rat was placed in the cylinder under the same conditions for 5 min and immobility time (the animal “freezes” in the water) was recorded.

| | | | | Test following |
|---|---|---|---|---|
| Group | n | Dose | Route | treatment |
| Water for Inj. | 10 | 4 mL/kg | i.p. | 30 min |
| Esketamine | 10 | 15 mg/kg | i.p. | |
| Esketamine | 10 | 30 mg/kg | i.p. | 30 min |
| D2-esketamine | 10 | 15 mg/kg | i.p. | 30 min |
| D2-esketamine | 10 | 30 mg/kg | i.p. | 30 min |
| D6-esketamine | 10 | 15 mg/kg | i.p. | 30 min |
| D6-esketamine | 10 | 30 mg/kg | i.p. | 30 min |
| ketamine | 10 | 30 mg/kg | i.p. | 30 min |

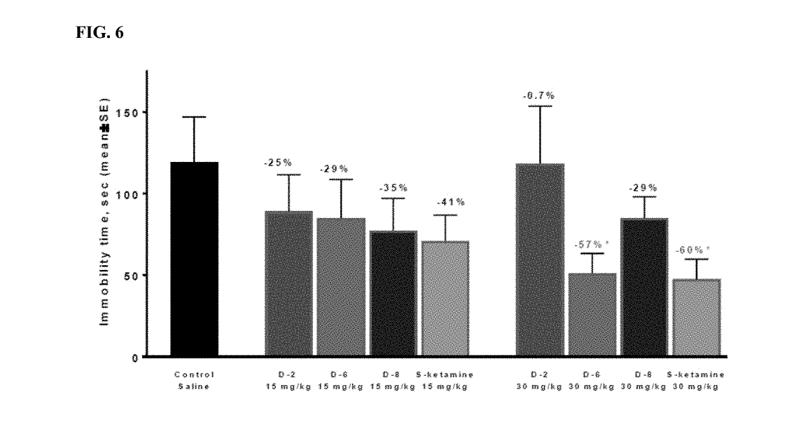

Results. The comparison in immobility time between the groups was done using One-Way Anova followed by Tukey post-hoc test. At a dose of 15 mg/kg, all the compounds showed comparable efficacy 25-41% reduction in immobility time (all non-significant). Following treatment with 30 mg/kg S-ketamine and D-6 S-ketamine, a further increment was observed in their antidepressant-like effect (60% and 57% reduction in immobility time, respectively, p<0.05). At this dose the effect of D-2 and D-8 S-ketamine were less pronounced compared to the lower dose. All the three deuterated compounds have a similar efficacy as Esketamine at the dose of 15 mg/kg in rat FST model. There were variations in the response of D-2 and D-8 S-ketamine at the higher 30 mg/kg dose. See FIG. 6.

### EXAMPLE 15

### Oral Single Dose PK Study in Rats

Male SD rats, 3 per timepoint, alternative timepoints, were used. Animals were dosed 15 mg/kg and 60 mg/kg with esketamine (DO), D2-esketamine, D6-esketamine, and D8-esketamine. Plasma was sampled at 10 min, 30 min, 1 h, 2 h, 3 h, 4 h, 7 h, 12 h, 24 h, and 30 h. The PK of the parent compound (deuterated and non-deuterated), as well as norketamine (deuterated and non-deuterated), 6-OH-norketamine (deuterated and non-deuterated), and dehydronorketamine (deuterated and non-deuterated) were analyzed.

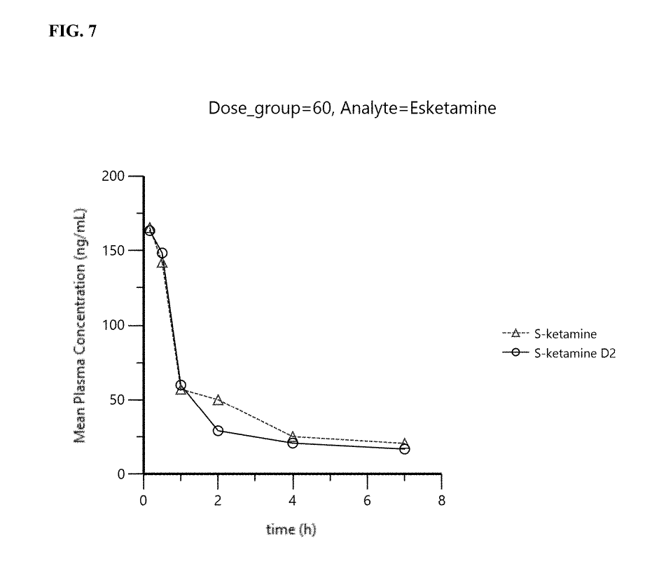

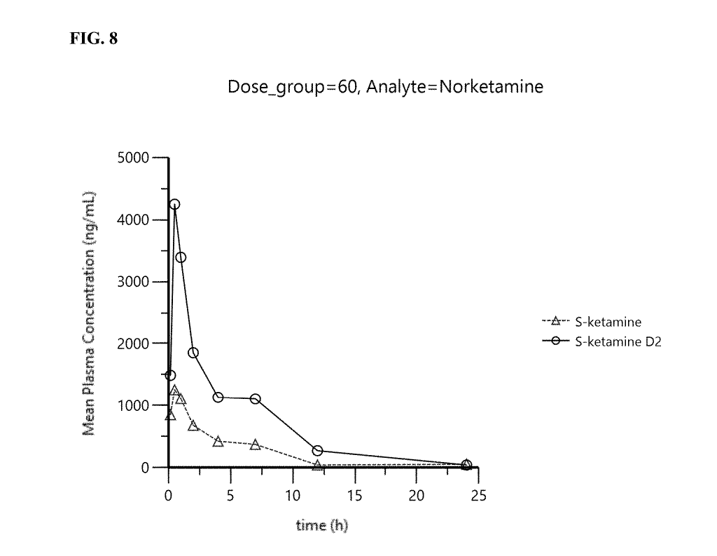

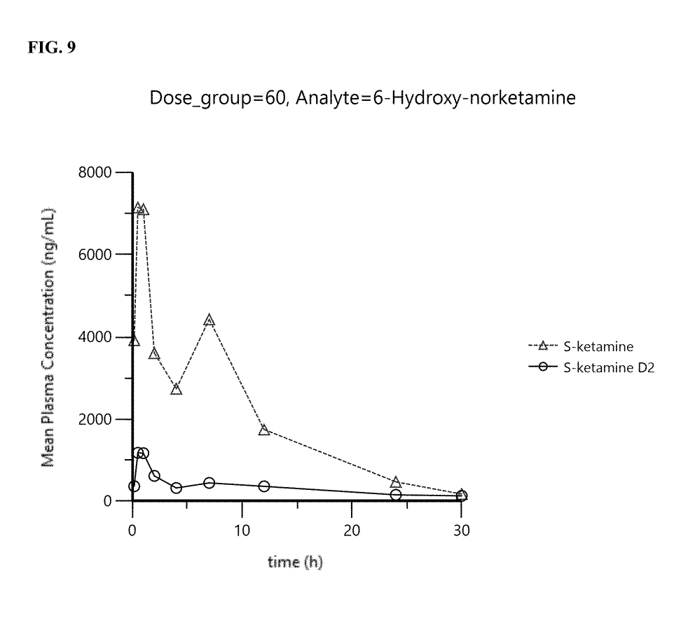

After single oral administration of 15 or 60 mg/kg of S-ketamine or S-ketamine D2 to male SD rats, the plasma exposure to the parent compound was essentially unchanged (see FIGS. 7-9).

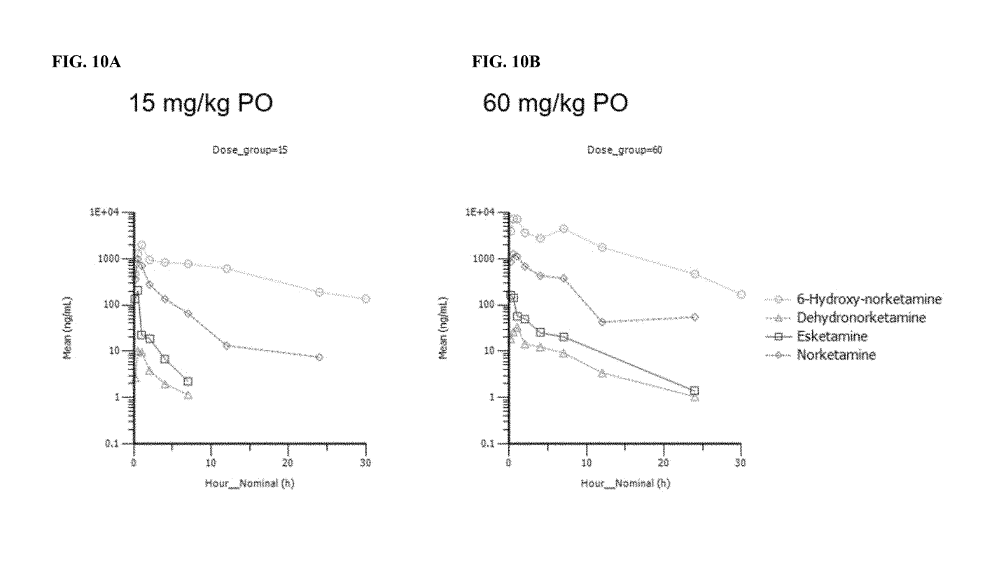

Esketamine's (DO) metabolism is fast, resulting in exposure predominantly to metabolites. The primary metabolite, norketamine, is further metabolized to 6-OH norketamine, the most prominent metabolite. Dehydronorketamine is a minor metabolite. See FIGS. 10A and 10B.

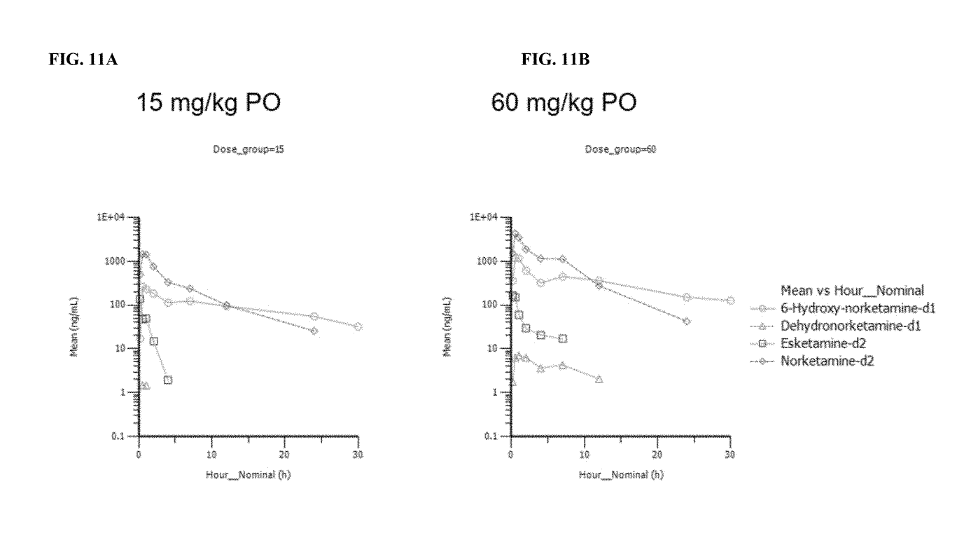

D2-esketamine exposure is shown in FIGS. 11A and 11B. Norketamine levels are higher and 6-OH norketamine levels are lower, as compared to esketamine. Dehydronorketamine is a minor metabolite.

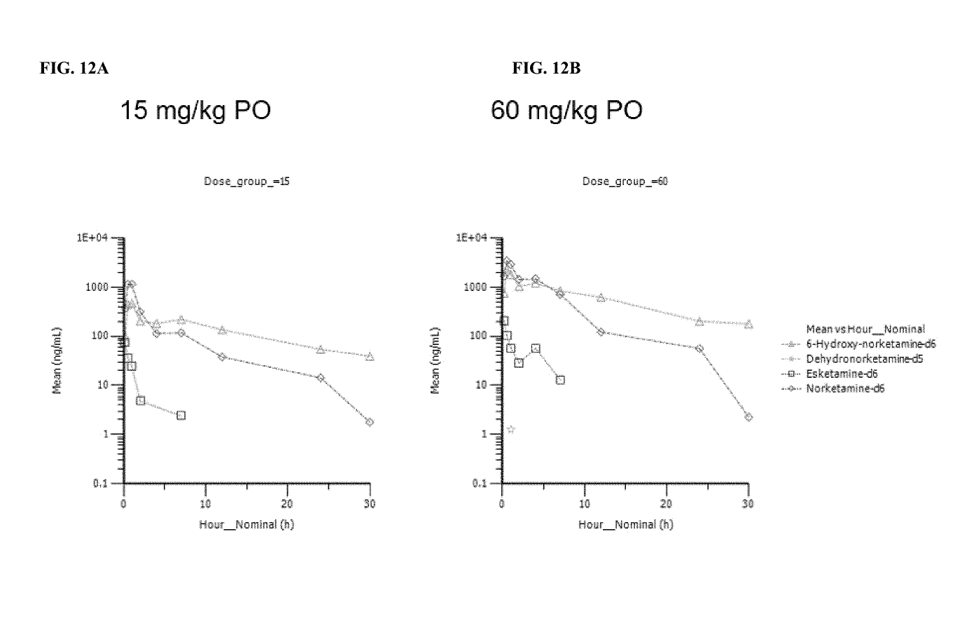

D6-esketamine exposure is shown in FIGS. 12A and 12B. Norketamine levels are higher and 6-OH-norketamine levels are lower, as compared to esketamine. Dehydronorketamine is not present.

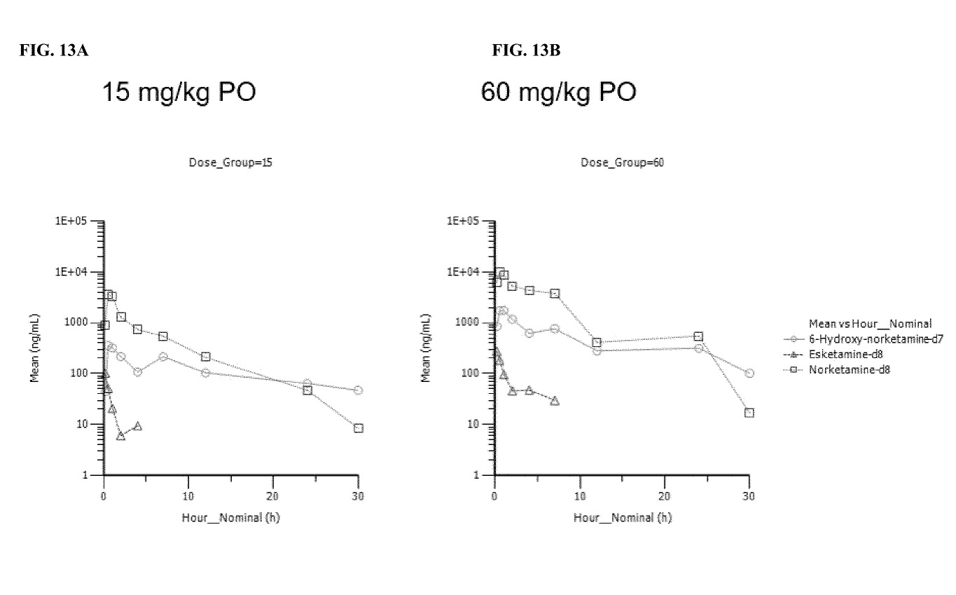

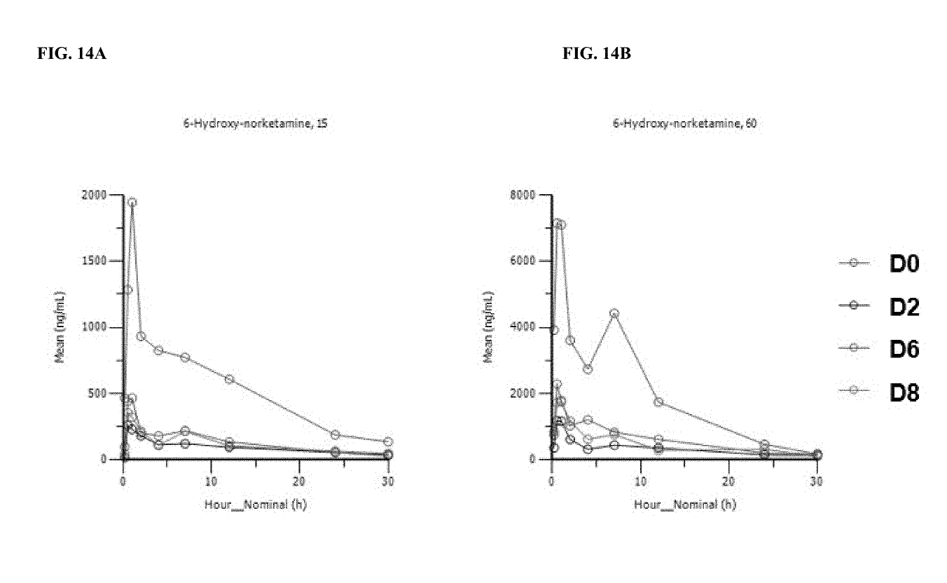

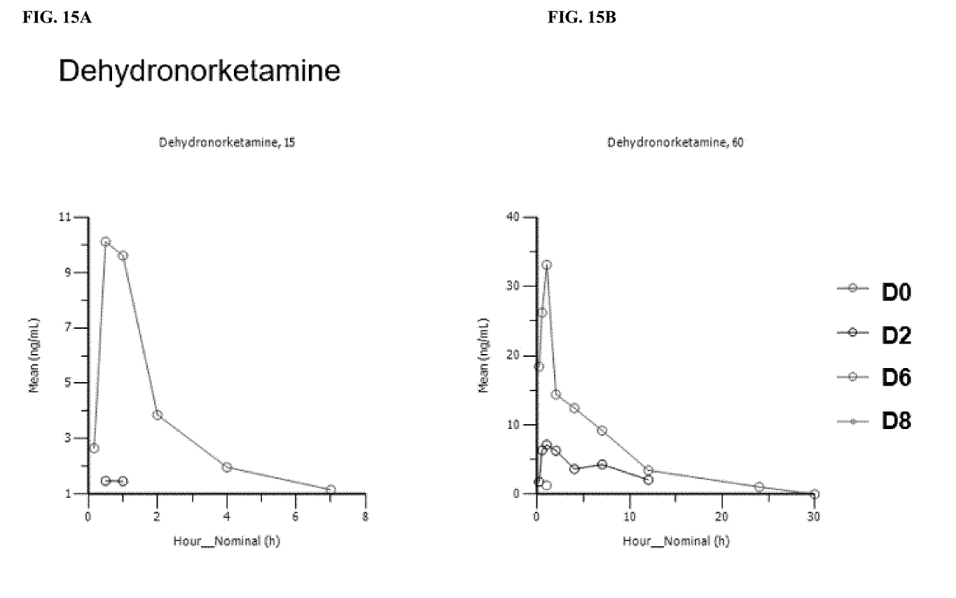

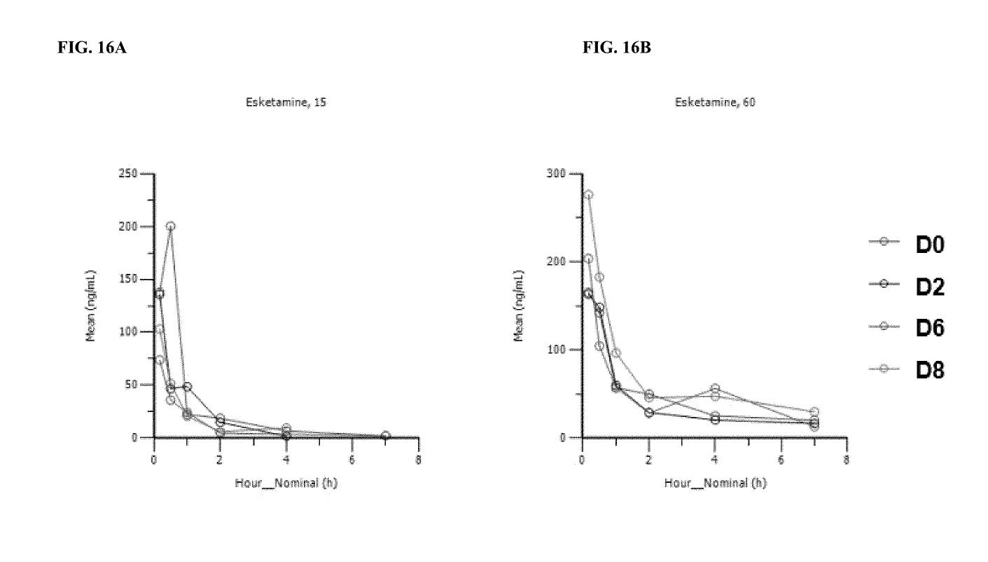

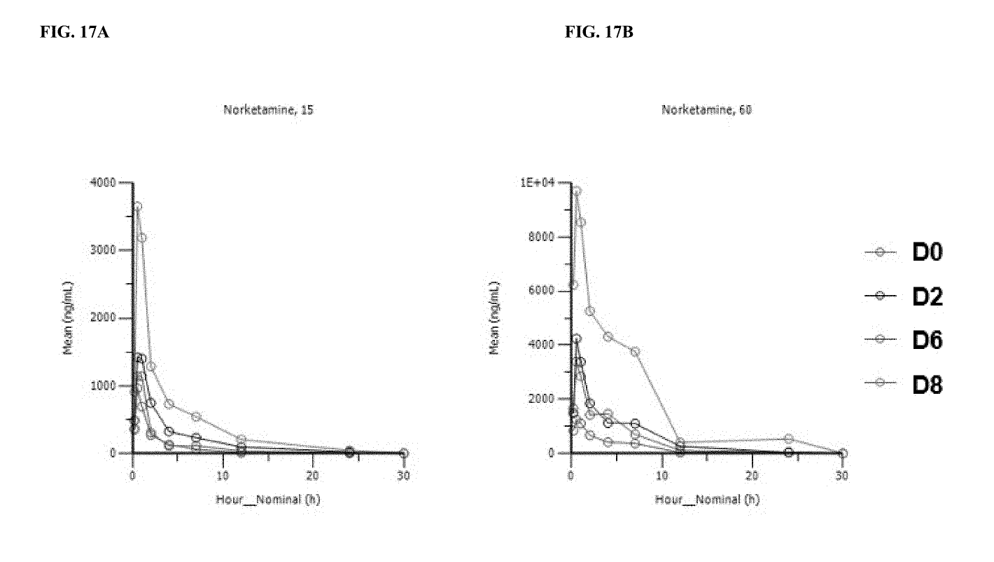

D8-esketamine exposure is shown in FIGS. 13A and 13B. D8-esketamine exhibited higher norketamine levels, as compared to esketamine. The 6-OH ketamine levels are similar to those observed for D2 esketamine and D6 esketamine. Dehydronorketamine is not present.

Additional PK information is provided in the following tables.

| | Dose | Cmax | Tmax | AUC0-t |
|---|---|---|---|---|
| | (mg/kg) | (ng/mL) | (min) | (ng * h/mL) |
| D0 | 15 | 200 | 30 | 183 |
| D2 | 15 | 136 | 10 | 114 |
| D6 | 15 | 74 | 10 | 72 |
| D8 | 15 | 103 | 10 | 82 |

| | Dose | Cmax | Tmax | AUC0-t |
|---|---|---|---|---|
| | (mg/kg) | (ng/mL) | (min) | (ng * h/mL) |
| D0 | 60 | 165 | 10 | 498 (311)* |
| D2 | 60 | 163 | 10 | 268 |
| D6 | 60 | 204 | 10 | 340 |
| D8 | 60 | 276 | 10 | 450 |

*24 hr time point excluded (7 hr was BLQ)

| | | AUC0-t (ng * h/mL) | | | |
|---|---|---|---|---|---|
| | Dose | | | 6-OH- | dehydro- |
| | (mg/kg) | Esketamine | Norketamine | norketamine | norketamine |
| D0 | 15 | 183 | 2180 | 15924 | 25 |
| D2 | 15 | 114 | 5643 | 2713 | 1 |
| D6 | 15 | 72 | 3095 | 3933 | — |
| D8 | 15 | 82 | 12308 | 3463 | — |

| | | AUC0-t (ng * h/mL) | | | |
|---|---|---|---|---|---|
| | Dose | | | 6-OH- | dehydro- |
| | (mg/kg) | Esketamine | Norketamine | norketamine | norketamine |
| D0 | 60 | 498 (311) | 5840 | 58707 | 165 |
| D2 | 60 | 268 | 17301 | 9677 | 49 |
| D6 | 60 | 341 | 14176 | 17963 | 1 |
| D8 | 60 | 450 | 54245 | 14119 | — |

Aspects of the Disclosure

Aspect 1. A compound of Formula I, or a pharmaceutically acceptable salt thereof:

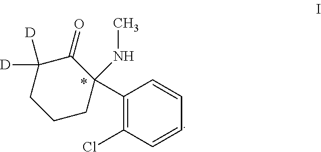 [SMILES: [2H]C1([2H])CCCC(NC)(c2ccccc2Cl)C1=O]

Aspect 2. The compound as recited in Aspect 1, wherein at least one position substituted with deuterium has deuterium enrichment of no less than about 98%.

Aspect 3. The compound as recited in Aspect 1, wherein at least one position substituted with deuterium has deuterium enrichment of no less than about 90%.

Aspect 4. The compound as recited in Aspect 1, wherein at least one position substituted with deuterium has deuterium enrichment of no less than about 50%.

Aspect 5. The compound as recited in Aspect 1, wherein at least one position substituted with deuterium has deuterium enrichment of no less than about 10%.

Aspect 6. A compound of any one of Aspects 1-5, wherein the carbon marked with an asterisk has (R)-configuration.

Aspect 7. A compound of any one of Aspects 1-5, wherein the carbon marked with an asterisk has (S)-configuration.

Aspect 8. A compound of any one of Aspects 1-7, wherein the pharmaceutically acceptable salt is a DCl salt.

Aspect 9. The compound which is:

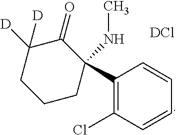

Aspect 10. A method for the treatment, prevention, or amelioration of one or more symptoms of a disorder selected from the group consisting of Rett syndrome, depression, major depressive disorder, refractory depression, suicidality, treatment resistant depression, obsessive-compulsive disorder, fibromyalgia, post-traumatic stress syndrome, autism spectrum disorder, and depression associated with genetic disorders, in a subject comprising administering a therapeutically effective amount of a compound of any one of Aspects 1-8.

Aspect 11. A method for the treatment, prevention, or amelioration of one or more symptoms of a disorder selected from the group consisting of Rett syndrome, depression, major depressive disorder, refractory depression, suicidality, treatment resistant depression, obsessive-compulsive disorder, fibromyalgia, post-traumatic stress syndrome, autism spectrum disorder, and depression associated with genetic disorders, in a subject comprising administering a therapeutically effective amount of a compound of Aspect 10.

Aspect 12. The method as recited in Aspect 10 or Aspect 11, further comprising the administration of another therapeutic agent.

Aspect 13. The method as recited in Aspect 10 or Aspect 11, wherein said compound has at least one of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

Aspect 14. The method as recited in Aspect 10 or Aspect 11, wherein said compound has at least two of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

Aspect 15. The method as recited in Aspect 10 or Aspect 11, wherein the method affects a decreased metabolism of the compound per dosage unit thereof by at least one polymorphically-expressed cytochrome P450 isoform in the subject, as compared to the corresponding non-isotopically enriched compound.

Aspect 16. The method as recited in Aspect 15, wherein the cytochrome P₄₅₀ isoform is selected from the group consisting of CYP2C8, CYP2C9, CYP2C19, and CYP2D6.

Aspect 17. The method as recited in Aspect 10 or Aspect 11, wherein said compound is characterized by decreased inhibition of at least one cytochrome P₄₅₀ or monoamine oxidase isoform in said subject per dosage unit thereof as compared to the non-isotopically enriched compound.

Aspect 18. The method as recited in Aspect 17, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

Aspect 19. The method as recited in Aspect 10 or Aspect 11, wherein the method affects the treatment of the disease while reducing or eliminating a deleterious change in a diagnostic hepatobiliary function endpoint, as compared to the corresponding non-isotopically enriched compound.

Aspect 20. The method as recited in Aspect 19, wherein the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

### REFERENCES

Sofia R D, Harakal J J. Arch Int Pharmacodyn Ther. 1975 March; 214(1): 68-74 Evaluation of ketamine HCl for anti-depressant activity.

Berman R M, Cappiello A, Anand A, Oren D A, Heninger G R, Charney D S, Krystal J H Biol Psychiatry 2000; 47: 351-354 Antidepressant Effects of Ketamine in Depressed Patients

Clements J A, Nimmo W S, Grant I S. J Pharm Sci. 1982 May; 71(5):539-42. Bioavailability, pharmacokinetics, and analgesic activity of ketamine in humans

Malinovsky J M, Servin F, Cozian A, Lepage J Y, Pinaud M. Br J Anaesth. 1996 August; 77(2):203-7. Ketamine and norketamine plasma concentrations after i.v., nasal and rectal administration in children.

Yanagihara Y, Ohtani M, Kariya S, Uchino K, Hiraishi T, Ashizawa N, Aoyama T, Yamamura Y, Yamada Y, Iga T. Biopharm Drug Dispos. 2003 January; 24(1):37-43. Plasma concentration profiles of ketamine and norketamine after administration of various ketamine preparations to healthy Japanese volunteers.

Peltoniemi M A, Saari T I, Hagelberg N M, Laine K, Kurkinen K J, Neuvonen P J, Olkkola K T Basic & Clinical Pharmacology & Toxicology, 2012, 111, 325-332 Rifampicin has a Profound Effect on the Pharmacokinetics of Oral S-Ketamine and Less on Intravenous S-Ketamine

Fanta S, Kinnunen M, Backman J T, Kalso E, Eur J Clin Pharmacol (2015) 71:441-447 Population pharmacokinetics of S-ketamine and norketamine in healthy volunteers after intravenous and oral dosing

Daly E J, Singh J B, Fedgchin M, Cooper K, Lim P, Shelton R C, Thase M E, Winokur A, Van Nueten L, Manji H, Drevets W C. JAMA Psychiatry. 2018 Feb. 1; 75(2):139-148 Efficacy and Safety of Intranasal Esketamine Adjunctive to Oral Antidepressant Therapy in Treatment-Resistant Depression: A Randomized Clinical Trial.

Ebert B, Mikkelsen S, Thorkildsen C, Borgbjerg F M. Eur J Pharmacol. 1997 Aug. 20; 333(1):99-104. Norketamine, the main metabolite of ketamine, is a non-competitive NMDA receptor antagonist in the rat cortex and spinal cord.

Oye I, Paulsen O, Maurset A. J Pharmacol Exp Ther. 1992 March; 260(3):1209-13 Effects of ketamine on sensory perception: evidence for a role of N-methyl-D-aspartate receptors.

Domino E F Anesthesiology 2010; 113:678-86 Taming the Ketamine Tiger

Zhang J C, Li S X, Hashimoto K. Pharmacol Biochem Behav. 2014 January; 116:137-41 R (−)-ketamine shows greater potency and longer lasting antidepressant effects than S (+)-ketamine.

Yang C, Shirayama Y, Zhang J C, Ren Q, Yao W, Ma M, Dong C, Hashimoto K. Transl Psychiatry. 2015 Sep. 1; 5:e632 R-ketamine: a rapid-onset and sustained antidepressant without psychotomimetic side effects.

Hashimoto K Psychol Med. 2016 August; 46(11):2449-51 Letter to the Editor: R-ketamine: a rapid-onset and sustained antidepressant without risk of brain toxicity.

Blonk M I, Koder B G, van den Bemt P M, Huygen F J. Eur J Pain. 2010 May; 14(5):466-72 Use of oral ketamine in chronic pain management: a review

Paslakis G, Gilles M, Meyer-Lindenberg A, Deuschle M. Pharmacopsychiatry. 2010 January; 43(1):33-5 Oral administration of the NMDA receptor antagonist S-ketamine as add-on therapy of depression: a case series.

Daly E J, Singh J B, Fedgchin M, Cooper K, Lim P, Melman C, Manji H, Van Nueten L, Shelton R C, Thase M E, Ahmad M, deBruecker G, Drevets W C. Neuropsychopharmacology (2015) 40, S340 Intranasal Esketamine in Treatment-resistant Depression, a Dose Response Study—Double Blind and Open Label Extension Data

The examples set forth above are disclosed to give a complete disclosure and description of how to make and use the claimed embodiments, and are not intended to limit the scope of what the inventors regard as what is disclosed herein. Modifications that are obvious are intended to be within the scope of the following claims. All publications, patents, and patent applications cited in this specification are incorporated herein by reference as if each such publication, patent or patent application were specifically and individually indicated to be incorporated herein by reference. However, with respect to any similar or identical terms found in both the incorporated publications or references and those expressly put forth or defined in this document, then those terms definitions or meanings expressly put forth in this document shall control in all respects.

What is claimed is:

1. A method of treating depression in a human comprising administering a therapeutically effect amount of a pharmaceutical composition comprising a compound of formula I 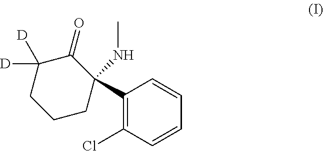 [SMILES: [2H]C1([2H])CCC[C@](NC)(c2ccccc2Cl)C1=O]
or a pharmaceutically acceptable salt thereof, or a mixture thereof;
to the human.

2. The method of claim 1, wherein the pharmaceutical composition comprises 20% (w/w) or less of a compound that is of the formula 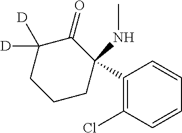 [SMILES: [2H]C1([2H])CCC[C@@](NC)(c2ccccc2Cl)C1=O]
based on the weight of the compound of formula I.

3. The method of claim 1, wherein each deuterium in the compound of formula I has a deuterium enrichment of no less than about 20%.

4. The method of claim 1, wherein the administration is oral, transdermal, intravenous, intranasal, or rectal administration.

5. The method of claim 1, wherein the depression is major depressive disorder, refractory depression, treatment resistant depression, or depression associate with a genetic disorder.

6. The method of claim 1, wherein the pharmaceutical salt of the compound of formula I is the DCl salt, the HCl salt, or a combination thereof.

7. The method of claim 1, further comprising administering another therapeutic agent for the treatment of depression to the human.

8. A pharmaceutical composition comprising a compound of formula I 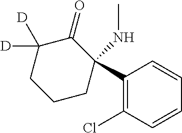 [SMILES: [2H]C1([2H])CCC[C@](NC)(c2ccccc2Cl)C1=O]
or a pharmaceutically acceptable salt thereof, or a mixture thereof;
and a pharmaceutically acceptable excipient.

9. The pharmaceutical composition of claim 8, comprising 20% (w/w) or less of a compound that is of the formula

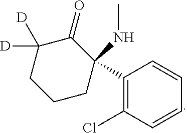 [SMILES: [2H]C1([2H])CCC[C@@](NC)(c2ccccc2Cl)C1=O]

10. The pharmaceutical composition of claim 8, wherein each deuterium in the compound of formula I has a deuterium enrichment of no less than about 20%.
